SEQUENCE LISTING (12 sequences)

SEQ ID NO: 1 - protein, 95 aa, Mycobacterium tuberculosis
MTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 2 - protein, 325 aa, Mycobacterium tuberculosis
  SIGNAL (1)..(40)
MTDVSRKIRAWGRRLMIGTAAAVVLPGLVGLAGGAATAGAFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 3 - protein, 404 aa, Artificial Sequence
  Recombinant Fusion protein Ag85B-ESAT-6
MATVNRSRHHHHHHHHIEGRSFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAGKLAMTEQQWNFAGIEAAASAIQGNVTSIHSLLDEGKQSLTKLAAAWGGSGSEAYQGVQQKWDATATELNNALQNLARTISEAGQAMASTEGNVTGMFA

SEQ ID NO: 4 - protein, 403 aa, Artificial Sequence
  Recombinant Fusion protein ESAT-6-Ag85B
MATVNRSRHHHHHHHHIEGRSMTEQQWNFAGIEAAASAIQGNVTSIHSLLDEGKQSLTKLAAAWGGSGSEAYQGVQQKWDATATELNNALQNLARTISEAGQAMASTEGNVTGMFAKLFSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 5 - DNA, 36 bp, Artificial Sequence
  Primer OPBR-4
ggcgccggcaagcttgccatgacagagcagcagtgg

SEQ ID NO: 6 - DNA, 26 bp, Artificial Sequence
  Primer OPBR-28
cgaactcgccggatcccgtgtttcgc

SEQ ID NO: 7 - DNA, 32 bp, Artificial Sequence
  Primer OPBR-48
ggcaaccgcgagatctttctcccggccggggc

SEQ ID NO: 8 - DNA, 27 bp, Artificial Sequence
  Primer OPBR-3
ggcaagcttgccggcgcctaacgaact

SEQ ID NO: 9 - DNA, 30 bp, Artificial Sequence
  Primer OPBR-75
ggacccagatctatgacagagcagcagtgg

SEQ ID NO: 10 - DNA, 47 bp, Artificial Sequence
  Primer OPBR-76
ccggcagccccggccgggagaaaagctttgcgaacatcccagtgacg

SEQ ID NO: 11 - DNA, 44 bp, Artificial Sequence
  Primer OPBR-77
gttcgcaaagcttttctcccggccggggctgccggtcgagtacc

SEQ ID NO: 12 - DNA, 20 bp, Artificial Sequence
  Primer OPBR-18
ccttcggtggatcccgtcag

(19) United States
(12) Patent Application Publication
(10) Pub. No.: US 2004/0013685 A1
(43) Pub. Date: Jan. 22, 2004

(54) Nucleic acid fragments and polypeptide fragments derived from M. tuberculosis

(76) Inventors: Peter Andersen, Bronshoj (DK); Rikke Louise Vinther Skjot, Hedehusene (DK); Li Mei Meng Okkels, Bagsvaerd (DK); Inger Brock, Dragor (DK); Thomas Oettinger, Hellerup (DK)

Correspondence Address: FROMMER LAWRENCE & HAUG, 745 FIFTH AVENUE- 10TH FL., NEW YORK, NY, 10151, US

(21) Appl. No.: 09/872,505

(22) Filed: Jun. 1, 2001

Related U.S. Application Data
(63) Continuation-in-part of application No. 09/804,980, filed on Mar. 13, 2001.
Application No. 09/804,980 is a continuation-in-part of application No. 09/246,191, filed on Dec. 30, 1998, now abandoned.
(63) Continuation-in-part of application No. 09/615,947, filed on Jul. 13, 2000.
(63) Continuation-in-part of application No. PCT/DK00/00398, filed on Jul. 13, 2000.
(60) Provisional application No. 60/070,488, filed on Jan. 5, 1998.
(60) Provisional application No. 60/144,011, filed on Jul. 15, 1999.

(30) Foreign Application Priority Data
Nov. 10, 1997 (DK) 1997 01277
Jul. 13, 1999 (DK) PA 1999 01020

(51) Int. Cl. A61K 39/02; C07H 21/04; C12P 21/04; C12N 1/21; C07K 14/35; C12N 15/74

(52) U.S. Cl. 424/190.1; 435/69.6; 435/252.3; 435/320.1; 530/350; 536/23.7

(57) ABSTRACT

The present invention is based on the identification and characterization of a number of novel M. tuberculosis derived proteins and protein fragments. The invention is directed to the polypeptides and immunologically active fragments thereof, the genes encoding them, immunological compositions such as vaccines and skin test reagents containing the polypeptide.

This application is a continuation-in-part of:

U.S. application Ser. No. 09/804,980 (attorney docket no. 670001-2002.4), filed Mar. 13, 2001, which is a continuation-in-part of U.S. application Ser. No. 09/246,191, filed Dec. 30, 1998, which claims priority from U.S. provisional 60/070,488, filed Jan. 5, 1998 and Danish patent application PA 1997 01277, filed Nov. 10, 1997;

U.S. application Ser. No. 09/615,947, filed Jul. 13, 2000, which claims priority from U.S. provisional 60/144,011, filed Jul. 15, 1999 and Danish patent application PA 1999 01020, filed Jul. 13, 1999; and

PCT application PCT/DK00/00398, filed Jul. 13, 2000, which claims priority from U.S. provisional 60/144,011, filed Jul. 15, 1999 and Danish patent application PA 1999 01020, filed Jul. 13, 1999, and is published Jan. 18, 2001 as WO01/04151.

Each of these patents, patent applications and patent publications, as well as all documents cited in the text of this application, and references cited in the documents referred to in this application (including references cited in the aforementioned patents, patent applications and patent publications or during their prosecution) are hereby incorporated herein by reference.

### FIELD OF INVENTION

The present invention discloses new immunogenic polypeptides and new immunogenic compositions based on polypeptides derived from the short time culture filtrate of M. tuberculosis.

### GENERAL BACKGROUND

Human tuberculosis caused by Mycobacterium tuberculosis (M. tuberculosis) is a severe global health problem, responsible for approx. 3 million deaths annually, according to the WHO. The worldwide incidence of new tuberculosis (TB) cases had been falling during the 1960s and 1970s but during recent years this trend has markedly changed in part due to the advent of AIDS and the appearance of multidrug resistant strains of M. tuberculosis.

The only vaccine presently available for clinical use is BCG, a vaccine whose efficacy remains a matter of controversy. BCG generally induces a high level of acquired resistance in animal models of TB, but several human trials in developing countries have failed to demonstrate significant protection. Notably, BCG is not approved by the FDA for use in the United States because BCG vaccination impairs the specificity of the Tuberculin skin test for diagnosis of TB infection.

This makes the development of a new and improved vaccine against TB an urgent matter, which has been given a very high priority by the WHO. Many attempts to define protective mycobacterial substances have been made, and different investigators have reported increased resistance after experimental vaccination. However, the demonstration of a specific long-term protective immune response with the potency of BCG has not yet been achieved.

Immunity to M. tuberculosis is characterized by some basic features; specifically sensitized T lymphocytes mediates protection, and the most important mediator molecule seems to be interferon gamma (IFN-γ).

M. tuberculosis holds, as well as secretes, several proteins of potential relevance for the generation of a new TB vaccine. For a number of years, a major effort has been put into the identification of new protective antigens for the development of a novel vaccine against TB. The search for candidate molecules has primarily focused on proteins released from dividing bacteria. Despite the characterization of a large number of such proteins only a few of these have been demonstrated to induce a protective immune response as subunit vaccines in animal models, most notably ESAT-6 and Ag85B (Brandt et al 2000).

In June 1998 Cole et al published the complete genome sequence of M. tuberculosis and predicted the presence of approximately 4000 open reading frames (Cole et al 1998). Following the sequencing of the M. tuberculosis genome, nucleotide sequences comprising Rv2653c, Rv2654c and Rv3873 are described in various databases and putative protein sequences for the above sequences are suggested, Rv2653c either comprising methionine or leucine as the first amino acid (The Sanger Centre database (http://www.sanger.ac.uk/Projects/M_{—}tuberculosis), Institut Pasteur database (http://genolist.pasteur.fr/TubercuList) and GenBank (http://www4.ncbi.nlm.nih.gov)).

However important, this sequence information cannot be used to predict if the DNA is translated and expressed as proteins in vivo. More importantly, it is not possible on the basis of the sequences to predict whether a given sequence will encode an immunogenic or an inactive protein. The only way to determine if a protein is recognized by the immune system during or after an infection with M. tuberculosis is to produce the given protein and test it in an appropriate assay as described herein. In WO00/11214, published Mar. 2, 2000, it is described that specific generic deletions can serve as markers to distinguish between avirulent and virulent mycobacteria strains.

Diagnosing M. tuberculosis infection in its earliest stage is important for effective treatment of the disease. Current diagnostic assays to determine M. tuberculosis infection are expensive and labour-intensive. In the industrialised part of the world the majority of patients exposed to M. tuberculosis receive chest x-rays and attempts are made to culture the bacterium in vitro from sputum samples. X-rays are insensitive as a diagnostic assay and can only identify infections in a very progressed stage. Culturing of M. tuberculosis is also not ideal as a diagnostic tool, since the bacteria grows poorly and slowly outside the body, which can produce false negative test results and take weeks before results are obtained. The standard tuberculin skin test is an inexpensive assay, used in third world countries, however it is far from ideal in detecting infection because it cannot distinguish M. tuberculosis-infected individuals from M. bovis BCG-vaccinated individuals and therefore cannot be used in areas of the world where patients receive or have received childhood vaccination with bacterial strains related to M. tuberculosis, e.g. a BCG vaccination.

Animal tuberculosis is caused by Mycobacterium bovis, which is closely related to M. tuberculosis and within the tuberculosis complex. M. bovis is an important pathogen that can infect a range of hosts, including cattle and humans. Tuberculosis in cattle is a major cause of economic loss and represents a significant cause of zoonotic infection. A number of strategies have been employed against bovine TB, but the approach has generally been based on government-organised programmes by which animals deemed positive to defined screening test are slaughtered. The most common test used in cattle is Delayed-type hypersensitivity with PPD as antigen, but alternative in vitro assays are also developed. However, investigations have shown that both the in vivo and the in vitro test have a relative low specificity, and the detection of false-positive is a significant economic problem (Pollock et al 2000). There is therefore a great need for a more specific diagnostic reagent, which can be used either in vivo or in vitro to detect M. bovis infections in animals.

### SUMMARY OF THE INVENTION

The invention is related to prevention, treatment and detection of infections caused by species of the tuberculosis complex (M. tuberculosis, M. bovis, M. africanum) by the use of a polypeptide comprising a M. tuberculosis antigen or an immunogenic portion or other variant thereof, or by the use of a DNA sequence encoding a M. tuberculosis antigen or an immunogenic portion or other variant thereof.

### DETAILED DISCLOSURE OF THE INVENTION

The present invention relates to a substantially pure polypeptide, which comprises an amino acid sequence selected from

(a) Rv2653c, Rv2654c or RD1-ORF5;

(b) an immunogenic portion, e.g. a T-cell epitope, of any one of the sequences in (a); and /or

(c) an amino acid sequence analogue having at least 70% sequence identity to any one of the sequences in (a) or (b) and at the same time being immunogenic.

Preferably, the amino acid sequence analogue has at least 80%, more preferred at least 90% and most preferred at least 95% sequence identity to any one of the sequences in (a) or (b).

The invention further relates to a fusion polypeptide, which comprises an amino acid sequence selected from

(a) Rv2653c, Rv2654c or RD1-ORF5;

(b) an immunogenic portion, e.g. a T-cell epitope, of any one of the sequences in (a); and /or

(c) an amino acid sequence analogue having at least 70% sequence identity to any one of the sequences in (a) or (b) and at the same time being immunogenic;

and at least one fusion partner.

Preferably, the fusion partner comprises a polypeptide fragment selected from

(a) a polypeptide fragment derived from a virulent mycobacterium, such as ESAT-6, MPB64, MPT64, TB10.4, CFP10, RD1-ORF5, RD1-ORF2, Rv1036, Ag85A, Ag85B, Ag85C, 19 kDa lipoprotein, MPT32, MPB59, Rv0285, Rv1195, Rv1386, Rv3878, MT3106.1 and alpha-crystallin;

(b) a polypeptide according to the invention and defined above and/or

(c) at least one immunogenic portion, e.g. a T-cell epitope, of any of such polypeptides in (a) or (b).

The invention further relates to a polypeptide, which comprises an amino acid sequence selected from

(a) Rv2653c, Rv2654c or RD1-ORF5;

(b) an immunogenic portion, e.g. a T-cell epitope, of any one of the sequences in (a); and /or

(c) an amino acid sequence analogue having at least 70% sequence identity to any one of the sequences in (a) or (b) and at the same time being immunogenic;

which is lipidated so as to allow a self-adjuvating effect of the polypeptide.

Further, the invention relates to a polypeptide, which comprises an amino acid sequence selected from

(a) Rv2653c, Rv2654c or RD1-ORF5;

(b) an immunogenic portion, e.g. a T-cell epitope, of any one of the sequences in (a); and /or

(c) an amino acid sequence analogue having at least 70% sequence identity to any one of the sequences in (a) or (b) and at the same time being immunogenic;

for use as a vaccine, as a pharmaceutical or as a diagnostic reagent.

In another embodiment, the invention relates to the use of a polypeptide as defined above or the preparation of a pharmaceutical composition for diagnosis, e.g. for diagnosis of tuberculosis caused by virulent mycobacteria, e.g. by Mycobacterium tuberculosis, Mycobacterium africanum or Mycobacterium bovis, and the use of a polypeptide as defined above for the preparation of a pharmaceutical composition, e.g. for the vaccination against infection caused by virulent mycobacteria, e.g. by Mycobacterium tuberculosis, Mycobacterium africanum or Mycobacterium bovis.

In a still further embodiment, the invention relates to an immunogenic composition comprising a polypeptide as defined above, preferably in the form of a vaccine or in the form of a skin test reagent.

In another embodiment, the invention relates to a nucleic acid fragment in isolated form which

(a) comprises a nucleic acid sequence which encodes a polypeptide as defined above, or comprises a nucleic acid sequence complementary thereto; or

(b) has a length of at least 10 nucleotides and hybridizes readily under stringent hybridization conditions with a nucleotide sequence selected from Rv2653c, Rv2654c or RD1-ORF5 nucleotide sequences or a sequence complementary thereto, or with a nucleotide sequence selected from a sequence in (a).

The nucleic acid fragment is preferably a DNA fragment. The fragment can be used as a pharmaceutical.

In one embodiment, the invention relates to a vaccine comprising a nucleic acid fragment according to the invention, optionally inserted in a vector, the vaccine effecting in vivo expression of antigen by an animal, including a human being, to whom the vaccine has been administered, the amount of expressed antigen being effective to confer substantially increased resistance to tuberculosis caused by virulent mycobacteria, e.g. by Mycobacterium tuberculosis, Mycobacterium africanum or Mycobacterium bovis, in an animal, including a human being.

In a further embodiment, the invention relates to the use of a nucleic acid fragment according to the invention for the preparation of a composition for the diagnosis of tuberculosis caused by virulent mycobacteria, e. g. by Mycobacterium tuberculosis, Mycobacterium africanum or Mycobacterium bovis, and the use of a nucleic acid fragment according to the invention for the preparation of a pharmaceutical composition for the vaccination against tuberculosis caused by virulent mycobacteria, e.g. by Mycobacterium tuberculosis, Mycobacterium africanum or Mycobacterium bovis.

In a still further embodiment, the invention relates to a vaccine for immunizing an animal, including a human being, against tuberculosis caused by virulent mycobacteria, e.g. by Mycobacterium tuberculosis, Mycobacterium africanum or Mycobacterium bovis, comprising as the effective component a non-pathogenic microorganism, wherein at least one copy of a DNA fragment comprising a DNA sequence encoding a polypeptide as defined above has been incorporated into the microorganism (e.g. placed on a plasmid or in the genome) in a manner allowing the microorganism to express and optionally secrete the polypeptide.

In another embodiment, the invention relates to a replicable expression vector, which comprises a nucleic acid fragment according to the invention, and a transformed cell harbouring at least one such vector.

In another embodiment, the invention relates to a method for producing a polypeptide as defined above, comprising

(a) inserting a nucleic acid fragment according to the invention into a vector which is able to replicate in a host cell, introducing the resulting recombinant vector into the host cell, culturing the host cell in a culture medium under conditions sufficient to effect expression of the polypeptide, and recovering the polypeptide from the host cell or culture medium;

(b) isolating the polypeptide from a whole mycobacterium, e.g. Mycobacterium tuberculosis, Mycobacterium africanum or Mycobacterium bovis, from culture filtrate or from lysates or fractions thereof; or

(c) synthesizing the polypeptide e.g. by solid or liquid phase peptide synthesis.

The invention also relates to a method of diagnosing tuberculosis caused by virulent mycobacteria, e.g. by Mycobacterium tuberculosis, Mycobacterium africanum or Mycobacterium bovis, in an animal, including a human being, comprising intradermally injecting, in the animal, a polypeptide as defined above or an immunogenic composition as defined above, a positive skin response at the location of injection being indicative of the animal having tuberculosis, and a negative skin response at the location of injection being indicative of the animal not having tuberculosis.

In another embodiment, the invention relates to a method for immunizing an animal, including a human being, against tuberculosis caused by virulent mycobacteria, e.g. by Mycobacterium tuberculosis, Mycobacterium africanum or Mycobacterium bovis, comprising administering to the animal the polypeptide as defined above, the immunogenic composition according to the invention, or the vaccine according to the invention.

Another embodiment of the invention relates to a monoclonal or polyclonal antibody, which is specifically reacting with a polypeptide as defined above in an immuno assay, or a specific binding fragment of said antibody. Preferably, said antibody is for use as a diagnostic reagent, e.g. for detection of mycobacterial antigens in sputum, urine or other body fluids of an infected animal, including a human being.

In a further embodiment the invention relates to a pharmaceutical composition which comprises an immunologically responsive amount of at least one member selected from the group consisting of:

(a) a polypeptide selected from Rv2653c, Rv2654c or RD1-ORF5, or an immunogenic portion thereof;

(b) an amino acid sequence which has a sequence identity of at least 70% to any one of said polypeptides in (a) and is immunogenic;

(c) a fusion polypeptide comprising at least one polypeptide or amino acid sequence according to (a) or (b) and at least one fusion partner;

(d) a nucleic acid sequence which encodes a polypeptide or amino acid sequence according to (a), (b) or (c);

(e) a nucleic acid sequence,which is complementary to a sequence according to (d);

(f) a nucleic acid sequence which has a length of at least 10 nucleotides and which hybridizes under stringent conditions with a nucleic acid sequence according to (d) or (e); and

(g) a non-pathogenic micro-organism which has incorporated (e.g. placed on a plasmid or in the genome) therein a nucleic acid sequence according to (d), (e) or (f) in a manner to permit expression of a polypeptide encoded thereby.

In a still further embodiment the invention relates to a method for stimulating an immunogenic response in an animal which comprises administering to said animal an immunologically stimulating amount of at least one member selected from the group consisting of:

(a) a polypeptide selected from Rv2653c, Rv2654c or RD1-ORF5, or an immunogenic portion thereof;

(b) an amino acid sequence which has a sequence identity of at least 70% to any one of said polypeptides in (a) and is immunogenic;

(c) a fusion polypeptide comprising at least one polypeptide or amino acid sequence according to (a) or (b) and at least one fusion partner;

(d) a nucleic acid sequence which encodes a polypeptide or amino acid sequence according to (a), (b) or (c);

(e) a nucleic acid sequence which is complementary to a sequence according to (d);

(f) a nucleic acid sequence which has a length of at least 10 nucleotides and which hybridizes under stringent conditions with a nucleic acid sequence according to (d) or (e); and

(g) a non-pathogenic micro-organism which has incorporated therein (e.g. placed on a plasmid or in the genome) a nucleic acid sequence according to (d), (e) or (f) in a manner to permit expression of a polypeptide encoded thereby.

The vaccine, immunogenic composition and pharmaceutical composition according to the invention can be used prophylactically in a subject not infected with a virulent mycobacterium; or therapeutically in a subject already infected with a virulent mycobacterium.

The invention also relates to a method for diagnosing previous or ongoing infection with a virulent mycobacterium, said method comprising

(a) contacting a sample, e.g. a blood sample, with a composition comprising an antibody according to the invention, a nucleic acid fragment according to the invention and/or a polypeptide as defined above, or

(b) contacting a sample, e.g. a blood sample comprising mononuclear cells (e.g. T-lymphocytes), with a composition comprising one or more polypeptides as defined above in order to detect a positive reaction, e.g. proliferation of the cells or release of cytokines such as IFN-γ.

Finally, the invention relates to a method of diagnosing Mycobacterium tuberculosis infection in a subject comprising:

(a) contacting a polypeptide as defined above with a bodily fluid of the subject; (b) detecting binding of a antibody to said polypeptide, said binding being an indication that said subject is infected by Mycobacterium tuberculosis or is susceptible to Mycobacterium tuberculosis infection.

Definitions

The word “polypeptide” in the present invention should have its usual meaning. That is an amino acid chain of any length, including a full-length protein, oligopeptides, short peptides and fragments thereof, wherein the amino acid residues are linked by covalent peptide bonds.

The polypeptide may be chemically modified by being glycosylated, by being lipidated (e.g. by chemical lipidation with palmitoyloxy succinimide as described by Mowat et al. 1991 or with dodecanoyl chloride as described by Lustig et al. 1976), by comprising prosthetic groups, or by containing additional amino acids such as e.g. a his-tag or a signal peptide.

Each polypeptide may thus be characterised by specific amino acids and be encoded by specific nucleic acid sequences. It will be understood that such sequences include analogues and variants produced by recombinant or synthetic methods wherein such polypeptide sequences have been modified by substitution, insertion, addition or deletion of one or more amino acid residues in the recombinant polypeptide and still be immunogenic in any of the biological assays described herein. Substitutions are preferably “conservative”. These are defined according to the following table. Amino acids in the same block in the second column and preferably in the same line in the third column may be substituted for each other. The amino acids in the third column are indicated in one-letter code.

| | | |
|---|---|---|
| ALIPHATIC | Non-polar | GAP |
| | | ILV |
| | Polar-uncharged | CSTM |
| | | NQ |
| | Polar-charged | DE |
| | | KR |
| AROMATIC | | HFWY |

A preferred polypeptide within the present invention is an immunogenic antigen from M. tuberculosis. Such antigen can for example be derived from M. tuberculosis and/or M. tuberculosis culture filtrate. Thus, a polypeptide comprising an immunogenic portion of one of the above antigens may consist entirely of the immunogenic portion, or may contain additional sequences. The additional sequences may be derived from the native M. tuberculosis antigen or be heterologous and such sequences may, but need not, be immunogenic.

Each polypeptide is encoded by a specific nucleic acid sequence. It will be understood that such sequences include analogues and variants hereof wherein such nucleic acid sequences have been modified by substitution, insertion, addition or deletion of one or more nucleic acid. Substitutions are preferably silent substitutions in the codon usage which will not lead to any change in the amino acid sequence, but may be introduced to enhance the expression of the protein.

In the present context the term “substantially pure polypeptide fragment” means a polypeptide preparation which contains at most 5% by weight of other polypeptide material with which it is natively associated (lower percentages of other polypeptide material are preferred, e.g. at most 4%, at most 3%, at most 2%, at most 1 %, and at most ½%). It is preferred that the substantially pure polypeptide is at least 96% pure, i.e. that the polypeptide constitutes at least 96% by weight of total polypeptide material present in the preparation, and higher percentages are preferred, such as at least 97%, at least 98%, at least 99%, at least 99.25%, at least 99.5%, and at least 99.75%. It is especially preferred that the polypeptide fragment is in “essentially pure form”, i.e. that the polypeptide fragment is essentially free of any other antigen with which it is natively associated, i.e. free of any other antigen from bacteria belonging to the tuberculosis complex or a virulent mycobacterium. This can be accomplished by preparing the polypeptide fragment by means of recombinant methods in a non-mycobacterial host cell as will be described in detail below, or by synthesizing the polypeptide fragment by the well-known methods of solid or liquid phase peptide synthesis, e.g. by the method described by Merrifield or variations thereof.

By the term “virulent mycobacterium” is understood a bacterium capable of causing the tuberculosis disease in an animal or in a human being. Examples of virulent mycobacteria are M. tuberculosis, M. africanum, and M. bovis. Examples of relevant animals are cattle, possums, badgers and kangaroos.

By “a TB patient” is understood an individual with culture or microscopically proven infection with virulent mycobacteria, and/or an individual clinically diagnosed with TB and who is responsive to anti-TB chemotherapy. Culture, microscopy and clinical diagnosis of TB are well known by any person skilled in the art.

By the term “PPD-positive individual” is understood an individual with a positive Mantoux test or an individual where PPD induces a positive in vitro recall response determined by release of IFN-γ.

By the term “delayed type hypersensitivity reaction” (DTH) is understood a T-cell mediated inflammatory response elicited after the injection of a polypeptide into, or application to, the skin, said inflammatory response appearing 72-96 hours after the polypeptide injection or application.

By the term “IFN-γ” is understood interferon-gamma. The measurement of IFN-γ is used as an indication of an immunological response.

By the terms “nucleic acid fragment” and “nucleic acid sequence” are understood any nucleic acid molecule including DNA, RNA, LNA (locked nucleic acids), PNA, RNA, dsRNA and RNA-DNA-hybrids. Also included are nucleic acid molecules comprising non-naturally occurring nucleosides. The term includes nucleic acid molecules of any length e.g. from 10 to 10000 nucleotides, depending on the use. When the nucleic acid molecule is for use as a pharmaceutical, e.g. in DNA therapy, or for use in a method for producing a polypeptide according to the invention, a molecule encoding at least one epitope is preferably used, having a length from about 18 to about 1000 nucleotides, the molecule being optionally inserted into a vector. When the nucleic acid molecule is used as a probe, as a primer or in antisense therapy, a molecule having a length of 10-100 is preferably used. According to the invention, other molecule lengths can be used, for instance a molecule having at least 12, 15, 21, 24, 27,,30, 33, 36, 39, 42, 50, 60, 70, 80, 90,100, 200, 300, 400, 500 or 1000 nucleotides (or nucleotide derivatives), or a molecule having at most 10000, 5000, 4000, 3000, 2000, 1000, 700, 500, 400, 300, 200,100, 50, 40, 30 or 20 nucleotides (or nucleotide derivatives). It should be understood that these numbers can be freely combined to produce ranges.

The term “stringent” when used in conjunction with hybridization conditions is as defined in the art, i.e. the hybridization is performed at a temperature not more than 15-20° C. under the melting point Tm, cf. Sambrook et al, 1989, pages 11.45-11.49. Preferably, the conditions are “highly stringent”, i.e. 5-10° C. under the melting point Tm.

Throughout this specification, unless the context requires otherwise, the word “comprise”, or variations thereof such as “comprises” or “comprising”, will be understood to imply the inclusion of a stated element or integer or group of elements or integers but not the exclusion of any other element or integer or group of elements or integers.

The term “sequence identity” indicates a quantitative measure of the degree of homology between two amino acid sequences of equal length or between two nucleotide sequences of equal length. The two sequences to be compared must be aligned to best possible fit possible with the insertion of gaps or alternatively, truncation at the ends of the protein sequences. The sequence identity can be calculated as

$$\frac{( {N}_{ref} - {N}_{dif} ) 100}{{N}_{ref}} ,$$

wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N_{dif}=2 and N_{ref}=8). A gap is counted as non-identity of the specific residue(s), i.e. the DNA sequence AGTGTC will have a sequence identity of 75% with the DNA sequence AGTCAGTC (N_{dif}=2 and N_{ref}=8). Sequence identity can alternatively be calculated by the BLAST program e.g. the BLASTP program (Pearson W. R and D. J. Lipman (1988))(www.ncbi.nlm.nih.gov/cgi-bin/BLAST). In one aspect of the invention, alignment is performed with the sequence alignment method ClustalW with default parameters as described by Thompson J., et al 1994, available at http://www2.ebi.ac.uk/clustalw/.

A preferred minimum percentage of sequence identity is at least 80%, such as at least 85%, at least 90%, at least 91 %, at least 92%, at least 93%, at least 94%, at least 95%, at least 96%, at least 97%, at least 98%, at least 99%, and at least 99.5%.

In a preferred embodiment of the invention, the polypeptide comprises an immunogenic portion of the polypeptide, such as an epitope for a B-cell or T-cell.

The immunogenic portion of a polypeptide is a part of the polypeptide, which elicits an immune response in an animal or a human being, and/or in a biological sample determined by any of the biological assays described herein. The immunogenic portion of a polypeptide may be a T-cell epitope or a B-cell epitope. Immunogenic portions can be related to one or a few relatively small parts of the polypeptide, they can be scattered throughout the polypeptide sequence or be situated in specific parts of the polypeptide. For a few polypeptides epitopes have even been demonstrated to be scattered throughout the polypeptide covering the full sequence (Ravn et al 1999).

In order to identify relevant T-cell epitopes which are recognised during an immune response, it is possible to use a “brute force” method: Since T-cell epitopes are linear, deletion mutants of the polypeptide will, if constructed systematically, reveal what regions of the polypeptide are essential in immune recognition, e.g. by subjecting these deletion mutants e.g. to the IFN-γ assay described herein. Another method utilises overlapping oligopeptides for the detection of MHC class II epitopes, preferably synthetic, having a length of e.g. 20 amino acid residues derived from the polypeptide. These peptides can be tested in biological assays (e.g. the IFN-γ assay as described herein) and some of these will give a positive response (and thereby be immunogenic) as evidence for the presence of a T cell epitope in the peptide. For the detection of MHC class I epitopes it is possible to predict peptides that will bind (Stryhn et al. 1996) and hereafter produce these peptides synthetic and test them in relevant biological assays e.g. the IFN-γ assay as described herein. The peptides preferably having a length of e.g. 8 to 11 amino acid residues derived from the polypeptide. B-cell epitopes can be determined by analysing the B cell recognition to overlapping peptides covering the polypeptide of interest as e.g. described in Harboe et al 1998.

Although the minimum length of a T-cell epitope has been shown to be at least 6 amino acids, it is normal that such epitopes are constituted of longer stretches of amino acids. Hence, it is preferred that the polypeptide fragment of the invention has a length of at least 7 amino acid residues, such as at least 8, at least 9, at least 10, at least 12, at least 14, at least 16, at least 18, at least 20, at least 22, at least 24, and at least 30 amino acid residues. Hence, in important embodiments of the inventive method, it is preferred that the polypeptide fragment has a length of at most 50 amino acid residues, such as at most 40, 35, 30, 25, and 20 amino acid residues. It should be understood that these numbers can be freely combined to produce ranges.

It is expected that the peptides having a length of between 10 and 20 amino acid residues will prove to be most efficient as MHC class 11 epitopes and therefore especially preferred lengths of the polypeptide fragment used in the inventive method are 18, such as 15, 14, 13, 12 and even 11 amino acid residues. It is expected that the peptides having a length of between 7 and 12 amino acid residues will prove to be most efficient as MHC class I epitopes and therefore especially preferred lengths of the polypeptide fragment used in the inventive method are 11, such as 10, 9, 8 and even 7 amino acid residues.

Immunogenic portions of polypeptides may be recognised by a broad part (high frequency) or by a minor part (low frequency) of the genetically heterogenic human population. In addition some immunogenic portions induce high immunological responses (dominant), whereas others induce lower, but still significant, responses (subdominant). High frequency><low frequency can be related to the immunogenic portion binding to widely distributed MHC molecules (HLA type) or even by multiple MHC molecules (Kilgus et al. 1991, Sinigaglia et al 1988).

In the context of providing candidate molecules for a new vaccine against tuberculosis, the subdominant epitopes are however as relevant as are the dominant epitopes since it has been shown (Olsen et al 2000) that such epitopes can induce protection regardless of being subdominant.

A common feature of the polypeptides of the invention is their capability to induce an immunological response as illustrated in the examples. It is understood that a variant of a polypeptide of the invention produced by substitution, insertion, addition or deletion is also immunogenic determined by any of the assays described herein.

An immune individual is defined as a person or an animal, which has cleared or controlled an infection with virulent mycobacteria or has received a vaccination with M. bovis BCG.

An immunogenic polypeptide is defined as a polypeptide that induces an immune response in a biological sample or an individual currently or previously infected with a virulent mycobacterium.

The immune response may be monitored by one of the following methods:

An in vitro cellular response is determined by release of a relevant cytokine such as IFN-γ, from lymphocytes withdrawn from an animal or human being currently or previously infected with virulent mycobacteria, or by detection of proliferation of these T cells. The induction being performed by the addition of the polypeptide or the immunogenic portion to a suspension comprising from 1×10⁵ cells to 3×10⁵ cells per well. The cells being isolated from either the blood, the spleen, the liver or the lung and the addition of the polypeptide or the immunogenic portion resulting in a concentration of not more than 20 μg per ml suspension and the stimulation being performed from two to five days. For monitoring cell proliferation the cells are pulsed with radioactive labeled Thymidine and after 16-22 hours of incubation detecting the proliferation by liquid scintillation counting. A positive response being a response more than background plus two standard deviations. The release of IFN-γ can be determined by the ELISA method, which is well known to a person skilled in the art. A positive response being a response more than background plus two standard deviations. Other cytokines than IFN-γ could be relevant when monitoring the immunological response to the polypeptide, such as IL-12, TNF-α, IL-4, IL-5, IL-10, IL-6, TGF-β. Another and more sensitive method for determining the presence of a cytokine (e.g. IFN-γ) is the ELISPOT method where the cells isolated from either the blood, the spleen, the liver or the lung are diluted to a concentration of preferable of 1 to 4×10⁶ cells /ml and incubated for 18-22 hrs in the presence of of the polypeptide or the immunogenic portion resulting in a concentration of not more than 20 μg per ml. The cell suspensions are hereafter diluted to 1 to 2×10⁶/ml and transferred to Maxisorp plates coated with anti-IFN-γ and incubated for preferably 4 to 16 hours. The IFN-γ producing cells are determined by the use of labelled secondary anti-IFN-γ antibody and a relevant substrate giving rise to spots, which can be enumerated using a dissection microscope. It is also a possibility to determine the presence of mRNA coding for the relevant cytokine by the use of the PCR technique. Usually one or more cytokines will be measured utilizing for example the PCR, ELISPOT or ELISA. It will be appreciated by a person skilled in the art that a significant increase or decrease in the amount of any of these cytokines induced by a specific polypeptide can be used in evaluation of the immunological activity of the polypeptide.

An in vitro cellular response may also be determined by the use of T cell lines derived from an immune individual or an M. tuberculosis infected person where the T cell lines have been driven with either live mycobacteria, extracts from the bacterial cell or culture filtrate for 10 to 20 days with the addition of IL-2. The induction being performed by addition of not more than 20 μg polypeptide per ml suspension to the T cell lines containing from 1×10⁵ cells to 3×10⁵ cells per well and incubation being performed from two to six days. The induction of IFN-γ or release of another relevant cytokine is detected by ELISA. The stimulation of T cells can also be monitored by detecting cell proliferation using radioactively labeled Thymidine as described above. For both assays a positive response being a response more than background plus two standard deviations.

An in vivo cellular response which may be determined as a positive DTH response after intradermal injection or local application patch of at most 100 μg of the polypeptide or the immunogenic portion to an individual who is clinically or subdlinically infected with a virulent Mycobacterium, a positive response having a diameter of at least 5 mm 72-96 hours after the injection or application.

An in vitro humoral response is determined by a specific antibody response in an immune or infected individual. The presence of antibodies may be determined by an ELISA technique or a Western blot where the polypeptide or the immunogenic portion is absorbed to either a nitrocellulose membrane or a polystyrene surface. The serum is preferably diluted in PBS from 1:10 to 1:100 and added to the absorbed polypeptide and the incubation being performed from 1 to 12 hours. By the use of labeled secondary antibodies the presence of specific antibodies can be determined by measuring the OD e.g. by ELISA where a positive response is a response of more than background plus two standard deviations or alternatively a visual response in a Western blot.

Another relevant parameter is measurement of the protection in animal models induced after vaccination with the polypeptide in an adjuvant or after DNA vaccination. Suitable animal models include primates, guinea pigs or mice, which are challenged with an infection of a virulent Mycobacterium. Readout for induced protection could be decrease of the bacterial load in target organs compared to non-vaccinated animals, prolonged survival times compared to non-vaccinated animals and diminished weight loss compared to non-vaccinated animals.

In general, M. tuberculosis antigens, and DNA sequences encoding such antigens, may be prepared using any one of a variety of procedures.

They may be purified as native proteins from the M. tuberculosis cell or culture filtrate by procedures such as those described above. Immunogenic antigens may also be produced recombinantly using a DNA sequence encoding the antigen, which has been inserted into an expression vector and expressed in an appropriate host. Examples of host cells are E. coli. The polypeptides or immunogenic portion hereof can also be produced synthetically having fewer than about 100 amino acids, and generally fewer than 50 amino acids and may be generated using techniques well known to those ordinarily skilled in the art, such as commercially available solid-phase techniques where amino acids are sequentially added to a growing amino acid chain.

In the construction and preparation of plasmid DNA encoding the polypeptide as defined for DNA vaccination a host strain such as E. coli can be used. Plasmid DNA can then be prepared from overnight cultures of the host strain carrying the plasmid of interest, and purified using e.g. the Qiagen Giga-Plasmid column kit (Qiagen, Santa Clarita, Calif., USA) including an endotoxin removal step. It is essential that plasmid DNA used for DNA vaccination is endotoxin free.

The immunogenic polypeptides may also be produced as fusion proteins, by which methods superior characteristics of the polypeptide of the invention can be achieved. For instance, fusion partners that facilitate export of the polypeptide when produced recombinantly, fusion partners that facilitate purification of the polypeptide, and fusion partners which enhance the immunogenicity of the polypeptide fragment of the invention are all interesting possibilities. Therefore, the invention also pertains to a fusion polypeptide comprising at least one polypeptide or immunogenic portion defined above and at least one fusion partner. The fusion partner can, in order to enhance immunogenicity, be another polypeptide derived from M. tuberculosis, such as of a polypeptide fragment derived from a bacterium belonging to the tuberculosis complex, such as ESAT-6, TB10.4, CFP10, RD1-ORF5, RD1-ORF2, Rv1036, MPB64, MPT64, Ag85A, Ag85B (MPT59), MPB59, Rv0285, Rv1195, Rv1386, Rv3878, MT3106.1, Ag85C, 19 kDa lipoprotein, MPT32 and alpha-crystallin, or at least one T-cell epitope of any of the above mentioned antigens ((Skjøt et al 2000; Danish Patent application PA 2000 00666; Danish Patent application PA 1999 01020; U.S. patent application Ser. No. 09/0505,739; Rosenkrands et al 1998; Nagai et al 1991). The invention also pertains to a fusion polypeptide comprising mutual fusions of two or more of the polypeptides (or immunogenic portions thereof) of the invention.

Other fusion partners, which could enhance the immunogenicity of the product, are lymphokines such as IFN-γ, IL-2 and IL-12. In order to facilitate expression and/or purification, the fusion partner can e.g. be a bacterial fimbrial protein, e.g. the pilus components pilin and papA; protein A; the ZZ-peptide (ZZ-fusions are marketed by Pharmacia in Sweden); the maltose binding protein; gluthatione S-transferase; β-galactosidase; or poly-histidine. Fusion proteins can be produced recombinantly in a host cell, which could be E. coli, and it is a possibility to induce a linker region between the different fusion partners.

Other interesting fusion partners are polypeptides, which are lipidated so that the immunogenic polypeptide is presented in a suitable manner to the immune system. This effect is e.g. known from vaccines based on the Borrelia burgdorferi OspA polypeptide as described in e.g. WO 96/40718 A or vaccines based on the Pseudomonas aeruginosa OprI lipoprotein (Cote-Sierra J 1998). Another possibility is N-terminal fusion of a known signal sequence and an N-terminal cystein to the immunogenic polypeptide. Such a fusion results in lipidation of the immunogenic polypeptide at the N-terminal cystein, when produced in a suitable production host.

Another part of the invention pertains to a vaccine composition comprising a polypeptide (or at least one immunogenic portion thereof) or fusion polypeptide according to the invention. In order to ensure optimum performance of such a vaccine composition it is preferred that it comprises an immunologically and pharmaceutically acceptable carrier, vehicle or adjuvant.

An effective vaccine, wherein a polypeptide of the invention is recognized by the animal, will in an animal model be able to decrease bacterial load in target organs, prolong survival times and/or diminish weight loss after challenge with a virulent Mycobacterium, compared to non-vaccinated animals.

Suitable carriers are selected from the group consisting of a polymer to which the polypeptide(s) is/are bound by hydrophobic non-covalent interaction, such as a plastic, e.g. polystyrene, or a polymer to which the polypeptide(s) is/are covalently bound, such as a polysaccharide, or a polypeptide, e.g. bovine serum albumin, ovalbumin or keyhole limpet haemocyanin. Suitable vehicles are selected from the group consisting of a diluent and a suspending agent. The adjuvant is preferably selected from the group consisting of dimethyldioctadecylammonium bromide (DDA), Quil A, poly I:C, aluminium hydroxide, Freund's incomplete adjuvant, IFN-γ, IL-2, IL-12, monophosphoryl lipid A (MPL), Treholose Dimycolate (TDM), Trehalose Dibehenate and muramyl dipeptide (MDP).

Preparation of vaccines which contain peptide sequences as active ingredients is generally well understood in the art, as exemplified by U.S. Pat. Nos. 4,608,251; 4,601,903; 4,599,231 and 4,599,230, all incorporated herein by reference.

Other methods of achieving adjuvant effect for the vaccine include use of agents such as aluminum hydroxide or phosphate (alum), synthetic polymers of sugars (Carbopol), aggregation of the protein in the vaccine by heat treatment, aggregation by reactivating with pepsin treated (Fab) antibodies to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed. Other possibilities involve the use of immune modulating substances such as cytokines or synthetic IFN-γ inducers such as poly I:C in combination with the above-mentioned adjuvants.

Another interesting possibility for achieving adjuvant effect is to employ the technique described in Gosselin et al., 1992 (which is hereby incorporated by reference herein). In brief, a relevant antigen such as an antigen of the present invention can be conjugated to an antibody (or antigen binding antibody fragment) against the Fcγ receptors on monocytes/macrophages.

The vaccines are administered in a manner compatible with the dosage formulation, and in such amount as will be therapeutically effective and immunogenic. The quantity to be administered depends on the subject to be treated, including, e.g., the capacity of the individual's immune system to mount an immune response, and the degree of protection desired. Suitable dosage ranges are of the order of several hundred micrograms active ingredient per vaccination with a preferred range from about 0.1 μg to 1000 μg, such as in the range from about 1 μg to 300 μg, and especially in the range from about 10 μg to 50 μg. Suitable regimens for initial administration and booster shots are also variable but are typified by an initial administration followed by subsequent inoculations or other administrations.

The manner of application may be varied widely. Any of the conventional methods for administration of a vaccine are applicable. These are believed to include oral application on a solid physiologically acceptable base or in a physiologically acceptable dispersion, parenterally, by injection or the like. The dosage of the vaccine will depend on the route of administration and will vary according to the age of the person to be vaccinated and, to a lesser degree, the size of the person to be vaccinated.

The vaccines are conventionally administered parenterally, by injection, for example, either subcutaneously or intramuscularly. Additional formulations which are suitable for other modes of administration include suppositories and, in some cases, oral formulations. For suppositories, traditional binders and carriers may include, for example, polyalkalene glycols or triglycerides; such suppositories may be formed from mixtures containing the active ingredient in the range of 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and advantageously contain 10-95% of active ingredient, preferably 25-70%.

In many instances, it will be necessary to have multiple administrations of the vaccine. Especially, vaccines can be administered to prevent an infection with virulent mycobacteria and/or to treat established mycobacterial infection. When administered to prevent an infection, the vaccine is given prophylactically, before definitive clinical signs or symptoms of an infection are present.

Due to genetic variation, different individuals may react with immune responses of varying strength to the same polypeptide. Therefore, the vaccine according to the invention may comprise several different polypeptides in order to increase the immune response. The vaccine may comprise two or more polypeptides or immunogenic portions, where all of the polypeptides are as defined above, or some but not all of the peptides may be derived from virulent mycobacteria. In the latter example, the polypeptides not necessarily fulfilling the criteria set forth above for polypeptides may either act due to their own immunogenicity or merely act as adjuvants.

The vaccine may comprise 1-20, such as 2-20 or even 3-20 different polypeptides or fusion polypeptides, such as 3-10 different polypeptides or fusion polypeptides.

The invention also pertains to a method for immunising an animal, including a human being, against TB caused by virulent mycobacteria, comprising administering to the animal the polypeptide of the invention, or a vaccine composition of the invention as described above, or a living vaccine described above.

The invention also pertains to a method for producing an immunologic composition according to the invention, the method comprising preparing, synthesising or isolating a polypeptide according to the invention, and solubilizing or dispersing the polypeptide in a medium for a vaccine, and optionally adding other M. tuberculosis antigens and/or a carrier, vehicle and/or adjuvant substance.

The nucleic acid fragments of the invention may be used for effecting in vivo expression of antigens, i.e. the nucleic acid fragments may be used in so-called DNA vaccines as reviewed in Ulmer et al 1993, which is included by reference.

Hence, the invention also relates to a vaccine comprising a nucleic acid fragment according to the invention, the vaccine effecting in vivo expression of antigen by an animal, including a human being, to whom the vaccine has been administered, the amount of expressed antigen being effective to confer substantially increased resistance to infections caused by virulent mycobacteria in an animal, including a human being.

The efficacy of such a DNA vaccine can possibly be enhanced by administering the gene encoding the expression product together with a DNA fragment encoding a polypeptide which has the capability of modulating an immune response.

One possibility for effectively activating a cellular immune response for a vaccine can be achieved by expressing the relevant antigen in a vaccine in a non-pathogenic microorganism or virus. Well-known examples of such microorganisms are Mycobacterium bovis BCG, Salmonella and Pseudomona and examples of viruses are Vaccinia Virus and Adenovirus.

Therefore, another important aspect of the present invention is an improvement of the living BCG vaccine presently available, wherein one or more copies of a DNA sequence encoding one or more polypeptide as defined above has been incorporated into the genome of the micro-organism in a manner allowing the micro-organism to express and secrete the polypeptide. The incorporation of more than one copy of a nucleotide sequence of the invention is contemplated to enhance the immune response.

Another possibility is to integrate the DNA encoding the polypeptide according to the invention in an attenuated virus such as the vaccinia virus or Adenovirus (Rolph et al 1997). The recombinant vaccinia virus is able to replicate within the cytoplasma of the infected host cell and the polypeptide of interest can therefore induce an immune response, which is envisioned to induce protection against TB.

The invention also relates to the use of a polypeptide or nucleic acid of the invention for use as therapeutic vaccines as have been described in the literature exemplified by D. Lowry (Lowry et al 1999). Antigens with therapeutic properties may be identified based on their ability to diminish the severity of M. tuberculosis infection in experimental animals or prevent reactivation of previous infection, when administered as a vaccine. The composition used for therapeutic vaccines can be prepared as described above for vaccines.

The invention also relates to a method of diagnosing TB caused by a virulent mycobacterium in an animal, including a human being, comprising intradermally injecting, in the animal, a polypeptide according to the invention, a positive skin response at the location of injection being indicative of the animal having TB, and a negative skin response at the location of injection being indicative of the animal not having TB.

When diagnosis of previous or ongoing infection with virulent mycobacteria is the aim, a blood sample comprising mononuclear cells (i.e. T-lymphocytes) from a patient could be contacted with a sample of one or more polypeptides of the invention. This contacting can be performed in vitro and a positive reaction could e.g. be proliferation of the T-cells or release of cytokines such as IFN-γ into the extracellular phase. It is also conceivable to contact a serum sample from a subject with a polypeptide of the invention, the demonstration of a binding between antibodies in the serum sample and the polypeptide being indicative of previous or ongoing infection.

The invention therefore also relates to an in vitro method for diagnosing ongoing or previous sensitisation in an animal or a human being with a virulent mycobacterium, the method comprising providing a blood sample from the animal or human being, and contacting the sample from the animal with the polypeptide of the invention, a significant release into the extracellular phase of at least one cytokine by mononuclear cells in the blood sample being indicative of the animal being sensitised. A positive response being a response more than release from a blood sample derived from a patient without the TB diagnosis plus two standard deviations. The invention also relates to the in vitro method for diagnosing ongoing or previous sensitisation in an animal or a human being with a virulent mycobacterium, the method comprising providing a blood sample from the animal or human being, and by contacting the sample from the animal with the polypeptide of the invention demonstrating the presence of antibodies recognizing the polypeptide of the invention in the serum sample.

The immunogenic composition used for diagnosing may comprise 1-20, such as 2-20 or even 3-20 different polypeptides or fusion polypeptides, such as 3-10 different polypeptides or fusion polypeptides.

The nucleic acid probes encoding the polypeptide of the invention can be used in a variety of diagnostic assays for detecting the presence of pathogenic organisms in a given sample. A method of determining the presence of mycobacterial nucleic acids in an animal, including a human being, or in a sample, comprising administering a nucleic acid fragment of the invention to the animal or incubating the sample with the nucleic acid fragment of the invention or a nucleic acid fragment complementary thereto, and detecting the presence of hybridised nucleic acids resulting from the incubation (by using the hybridisation assays which are well-known in the art), is also included in the invention. Such a method of diagnosing TB might involve the use of a composition comprising at least a part of a nucleotide sequence as defined above and detecting the presence of nucleotide sequences in a sample from the animal or human being to be tested which hybridise with the nucleic acid fragment (or a complementary fragment) by the use of PCR technique.

A monoclonal or polyclonal antibody, which is specifically reacting with a polypeptide of the invention in an immuno assay, or a specific binding fragment of said antibody, is also a part of the invention. The antibodies can be produced by methods known to the person skilled in the art. Polyclonal antibodies can be raised in a mammal, for example, by one or more injections of a polypeptide according to the present invention and, if desired, an adjuvant. The monoclonal antibodies according to the present invention may, for example, be produced by the hybridoma method first described by Kohler and Milstein (1975), or may be produced by recombinant DNA methods such as described in U.S. Pat. No. 4,816,567. The monoclonal antibodies may also be isolated from phage libraries generated using the techniques described by McCafferty et al (1990), for example. Methods for producing antibodies are described in the literature, e.g. in U.S. Pat. No. 6,136,958.

A sample of a potentially infected organ may be contacted with such an antibody recognizing a polypeptide of the invention. The demonstration of the reaction by means of methods well known in the art between the sample and the antibody will be indicative of an ongoing infection. It is of course also a possibility to demonstrate the presence of anti-mycobacterial antibodies in serum by contacting a serum sample from a subject with at least one of the polypeptide fragments of the invention and using well-known methods for visualising the reaction between the antibody and antigen.

In diagnostics, an antibody, a nucleic acid fragment and/or a polypeptide of the invention can be used either alone, or as a constituent in a composition. Such compositions are known in the art, and comprise compositions in which the antibody, the nucleic acid fragment or the polypeptide of the invention is coupled, preferably covalently, to at least one other molecule, e.g. a label (e.g. radioactive or fluorescent) or a carrier molecule.

Concordance list

| | Protein | DNA | |
|---|---|---|---|
| | SEQ ID NO: | SEQ ID NO: | Synonyms |
| Rv2653c | 2 | | |
| Rv2654c | 4 | 3 | |
| RD1-ORF5 | 6 | 5 | Rv3873 (lacks |
| | | | 3 amino acids |
| | | | N-terminally) |
| Rv2653c-p1 | 7 | | |
| Rv2653c-p2 | 8 | | |
| Rv2653c-p3 | 9 | | |
| Rv2653c-p4 | 10 | | |
| Rv2653c-p5 | 11 | | |
| Rv2653c-p6 | 12 | | |
| Rv2653c-p7 | 13 | | |
| Rv2653c-p8 | 14 | | |
| Rv2653c-p9 | 15 | | |
| Rv2653c-p10 | 16 | | |
| Rv2654c-p1 | 17 | | |
| Rv2654c-p2 | 18 | | |
| Rv2654c-p3 | 19 | | |
| Rv2654c-p4 | 20 | | |
| Rv2654c-p5 | 21 | | |
| Rv2654c-p6 | 22 | | |
| RD1-ORF5-p1 | 23 | | |
| RD1-ORF5-p2 | 24 | | |
| RD1-ORF5-p3 | 25 | | |
| RD1-ORF5-p4 | 26 | | |
| RD1-ORF5-p5 | 27 | | |
| RD1-ORF5-p6 | 28 | | |
| RD1-ORF5-p7 | 29 | | |
| RD1-ORF5-p8 | 30 | | |
| RD1-ORF5-p9 | 31 | | |
| RD1-ORF5-p10 | 32 | | |
| RD1-ORF5-p11 | 33 | | |
| RD1-ORF5-p12 | 34 | | |
| RD1-ORF5-p13 | 35 | | |
| RD1-ORF5-p14 | 36 | | |
| RD1-ORF5-p15 | 37 | | |
| RD1-ORF5-p16 | 38 | | |
| RD1-ORF5-p17 | 39 | | |
| RD1-ORF5-p18 | 40 | | |
| RD1-ORF5-p19 | 41 | | |
| RD1-ORF5-p20 | 42 | | |
| RD1-ORF5-p21 | 43 | | |
| RD1-ORF5-p22 | 44 | | |
| RD1-ORF5-p23 | 45 | | |
| RD1-ORF5-p24 | 46 | | |
| RD1-ORF5-p25 | 47 | | |
| RD1-ORF5-p26 | 48 | | |
| RD1-ORF5-p27 | 49 | | |
| RD1-ORF5-p28 | 50 | | |
| RD1-ORF5-p29 | 51 | | |
| RD1-ORF5-p30 | 52 | | |
| RD1-ORF5-p31 | 53 | | |
| RD1-ORF5-p32 | 54 | | |
| RD1-ORF5-p36 | 55 | | |
| RD1-ORF5-p33 | 56 | | |
| RD1-ORF5-p34 | 57 | | |
| RD1-ORF5-p35 | 58 | | |
| PA2653c | | 59 | |
| PB2653c | | 60 | |
| PA2654c | | 61 | |
| PB2654c | | 62 | |
| Rv2653-F | | 63 | |
| Rv2653-R | | 64 | |
| Rv2654-F | | 65 | |
| Rv2654-R | | 66 | |
| RD1-ORF5f | | 67 | |
| RD1-ORF5r | | 68 | |

### EXAMPLES

### Example 1

Identification of Antigens, which are not Expressed in BCG Strains

In an effort to control the treat of TB, attenuated bacillus Calmette-Guerin (BCG) has been used as a live attenuated vaccine. BCG is an attenuated derivative of a virulent Mycobacterium bovis. The original BCG from the Pasteur Institute in Paris, France was developed from 1908 to 1921 by 231 passages in liquid culture and has never been shown to revert to virulence in animals, indicating that the attenuating mutation(s) in BCG are stable deletions and/or multiple mutations which do not readily revert. While physiological differences between BCG and M. tuberculosis and M. bovis has been noted, the attenuating mutations which arose during serial passage of the original BCG strain has been unknown until recently. The first mutations described are the loss of the gene encoding MPB64 in some BCG strains (Li et al., 1993, Oeftinger and Andersen, 1994) and the gene encoding ESAT-6 in all BCG strain tested (Harboe et al., 1996), later 3 large deletions in BCG have been identified (Mahairas et al., 1996). The region named RD1 includes the gene encoding ESAT-6 and an other (RD2) the gene encoding MPT64. Both antigens have been shown to have diagnostic potential and ESAT-6 has been shown to have properties as a vaccine candidate (cf. PCT/DK94/00273 and PCT/DK/00270). In order to find new M. tuberculosis specific diagnostic antigens as well as antigens for a new vaccine against TB, the RD1 region (17.499 bp) of M. tuberculosis H37Rv has been analyzed for Open Reading Frames (ORF). ORFs with a minimum length of 96 bp have been predicted using the algorithm described by Borodovsky and McIninch (1993), in total 27 ORFs have been predicted, 20 of these have possible diagnostic and/or vaccine potential, as they are deleted from all known BCG strains. The predicted ORFs include ESAT-6 (RD1-ORF7) and CFP10 (RD1-ORF6) described previously (Sørensen et al., 1995), as a positive control for the ability of the algorithm. In the present example is described the potential of predicted antigens for diagnosis of TB as well as potential as candidates for a new vaccine against TB.

Seven open reading frames (ORF) from the 17,499 kb RD1 region (Accession no. U34848) with possible diagnostic and vaccine potential have been identified and cloned by the present inventors. Identification and cloning of ORF rd1-orf5 is described below.

Identification of the ORF rd1-orf5.

The nucleotide sequence of rd1-orf5from M. tuberculosis H37Rv is set forth in SEQ ID NO: 5. The deduced amino acid sequence of RD1-ORF5 is set forth in SEQ ID NO: 6.

The DNA sequence rd1-orf5 contained an open reading frame starting with a GTG codon at position 3128-3130 and ending with a termination codon (TGA) at position 4241-4243 (position numbers referring to the location in RD1). The deduced amino acid sequence contains 371 residues corresponding to a molecular weight of 37,647.

Cloning of the ORF rd1-orf5.

The ORF rd1-orf5 was PCR cloned in the pQE32 (QIAGEN) expression vector. Preparation of oligonucleotides and PCR amplification of the rd1-orf5 encoding gene was carried out as described in example 2 in WO 99/24577 (corresponding to U.S. Ser. No. 09/246,191). Chromosomal DNA from M. tuberculosis H37Rv was used as template in the PCR reactions. Oligonucleotides were synthesized on the basis of the nucleotide sequence from the RD1 region (Accession no. U34848). The oligonucleotide primers were engineered to include a restriction enzyme site at the 5′ end and at the 3′ end by which a later subcloning was possible. Primers are listed in TABLE 1.

rd1-off5. A BamHI site was engineered immediately 5′ of the first codon of rd1-ORF5, and a HindIII site was incorporated right after the stop codon at the 3′ end. The gene rd1-ORF5 was subcloned in pQE32, giving pTO88.

The PCR fragments were digested with the suitable restriction enzymes, purified from an agarose gel and cloned into pQE-32. The construct was used to transform the E. coli XL1-Blue. Endpoints of the gene fusions were determined by the dideoxy chain termination method. Both strands of the DNA were sequenced.

Purification of recombinant RD1-ORF5.

The rRD1-ORF5 was fused N-terminally to the (His)₆-tag. Recombinant antigen was prepared as described in example 2 in WO 99/24577 (corresponding to U.S. Ser. No. 09/246,191), using a single colony of E. coli harbouring the pTO88 for inoculation. Purification of re-combinant antigen by Ni²⁺ affinity chromatography was also carried out as described in example 2 in WO 99/24577 (corresponding to U.S. Ser. No. 09/246,191). Fractions containing purified His-rRD1-ORF5 were pooled. The His-rRD1-ORF's were extensively dialysed against 10 mM Tris/HCl, pH 8.5, 3 M urea followed by an additional purification step performed on an anion exchange column (Mono Q) using fast protein liquid chromatography (FPLC) (Pharmacia, Uppsala, Sweden). The purification was carried out in 10 mM Tris/HCl, pH 8.5, 3 M urea and protein was eluted by a linear gradient of NaCl from 0 to 1 M. Fractions containing the His-rRD1-ORF were pooled and subsequently dialysed extensively against 25 mM Hepes, pH 8.0 before use.

TABLE 1

Sequence of the rd1-orf5 oligonucleotides^{a}.

| | | |
|---|---|---|
| Orientation and | | Position |
| oligonucleotide | Sequences (5′→3′) | (nt) |

Sense

| | | |
|---|---|---|
| RD1-ORF5f | CTGGGGATCCGCGTGATGACCAT- | 3028-3045 |
| | GCTGTGG | |

Antisense

| | | |
|---|---|---|
| RD1-ORF5r | TGCAAGCTTTCACCAGTCGTCCT- | 4243-4223 |
| | CTTCGTC | |

^{a}The oligonucleotides were constructed from the Accession number U34484 nucleotide sequence (Mahairas et al, 1996). Nucleotides (nt) underlined are not contained in the nucleotide sequence of RD1-ORF5. The positions correspond to the nucleotide sequence of Accession number U34484.

### Example 2

Biological Activity of the Purified Antigens

The recognition of the purified antigens in the mouse model of memory immunity to TB (described in example 1 in WO 99/24577 (corresponding to U.S. Ser. No. 09/246,191)) was investigated.

Interferon-γ Induction in the Mouse Model of TB Infection

Primary infections. 8 to 12 weeks old female C57BL/6j(H-2^{b}), CBA/J(H-2^{k}), DBA.2(H-2^{d}) and A.SW(H-2^{s}) mice (Bomholtegaard, Ry) were given intravenous infections via the lateral tail vein with an inoculum of 5×10⁴ M. tuberculosis suspended in PBS in a vol. of 0.1 ml. 14 days postinfection the animals were sacrificed and spleen cells were isolated and tested for the recognition of recombinant antigen.

As shown in TABLE 2, RD1-ORF5 gave rise to an IFN-γ release in two mice strains at a level corresponding to ⅔ of the response after stimulation with ST-CF.

Memory responses. 8-12 weeks old female C57BL/6j(H-2^{b}) mice (Bomholtegaard, Ry) were given intravenous infections via the lateral tail vein with an inoculum of 5×10⁴ M. tuberculosis suspended in PBS in a vol. of 0.1 ml. After 1 month of infection the mice were treated with isoniazid (Merck and Co., Rahway, N.J.) and rifabutin (Farmatalia Carlo Erba, Milano, Italy) in the drinking water, for two months. The mice were rested for 4-6 months before being used in experiments. For the study of the recall of memory immunity, animals were infected with an inoculum of 1×10⁶ bacteria i.v. and sacrificed at day 4 postinfection. Spleen cells were isolated and tested for the recognition of recombinant antigen.

As shown in TABLE 3, IFN-γ release after stimulation with RD1-ORF5 resulted in an IFN-γ release of approximately ⅓ of the response seen with ST-CF.

TABLE 2
T cell responses in primary TB infection.

| | C57BI/6j | DBA.2 | CBA/J | A.SW |
|---|---|---|---|---|
| Name | (H2^{b}) | (H2^{d}) | (H2^{k}) | (H2^{s}) |
| RD1-ORF5 | + | + | ++ | ++ |

Mouse IFN-γ release 14 days after primary infection with M. tuberculosis.
−: no response;
+: ⅓ of ST-CF;
++: ⅔ of ST-CF;
+++: level of ST-CF.
n.d. = not determined.

TABLE 3
T cell responses in memory immune animals.

| Name | Memory response |
|---|---|
| RD1-ORF5 | + |

Mouse IFN-γ release during recall of memory immunity to M. tuberculosis.
−: no response;
+: ⅓ of ST-CF;
++: ⅔ of ST-CF;
+++: level of ST-CF.

Interferon-γ Induction in Human TB Patients and BCG Vaccinated People.

Human donors: PBMC were obtained from healthy BCG vaccinated donors with no known exposure to patients with TB and from patients with culture or microscopy proven infection with Mycobacterium tuberculosis. Blood samples were drawn from the TB patients 1-4 months after diagnosis.

Lymphocyte preparations and cell culture: PBMC were freshly isolated by gradient centrifugation of heparinized blood on Lymphoprep (Nycomed, Oslo, Norway). The cells were resuspended in complete medium: RPMI 1640 (Gibco, Grand Island, N.Y.) supplemented with 40 μg/ml streptomycin, 40 U/ml penicillin, and 0.04 mM/ml glutamine, (all from Gibco Laboratories, Paisley, Scotland) and 10% normal human ABO serum (NHS) from the local blood bank. The number and the viability of the cells were determined by trypan blue staining. Cultures were established with 2.5×10⁵ PBMC in 200 μl in microtitre plates (Nunc, Roskilde, Denmark) and stimulated with no antigen, ST-CF, PPD (2.5 μg/ml), antigen in a final concentration of 5 μg/ml. Phytohaemagglutinin, 1 μg/ml (PHA, Difco laboratories, Detroit, Mich. was used as a positive control. Supernatants for the detection of cytokines were harvested after 5 days of culture, pooled and stored at −80° C. until use.

Cytokine analysis: Interferon-γ (IFN-γ) was measured with a standard ELISA technique using a commercially available pair of mAb's from Endogen and used according to the instructions for use. Recombinant IFN-γ (Gibco laboratories) was used as a standard. The detection level for the assay was 50 pg/ml. The variation between the duplicate wells did not exceed 10% of the mean.

As is seen from Table 4, RD1-ORF5 gives rise to IFN-γ responses close to the level of ST-CF. Between 60% and 90% of the donors show high IFN-γ responses (>1000 pg/ml).

TABLE 4
Results from the stimulation of human blood cells from 10
healthy BCG vaccinated or non vaccinated ST-CF responsive
healthy donors and 10 Tb patients with recombinant antigen
are shown. ST-CF, PPD and PHA are included for comparison.
Results are given in pg.
IFN-γ/ml and negative values below 300 pg/ml are shown as
“<”. nd = not done. Controls, Healthy BCG vaccinated,
or non vaccinated ST-CF positive

| Donor | no ag | PHA | PPD | STCF | RD1-ORF5 |
|---|---|---|---|---|---|
| 10 | < | nd | 3500 | 4200 | 690 |
| 11 | < | nd | 5890 | 4040 | 9030 |
| 12 | < | nd | 6480 | 3330 | 3320 |
| 13 | < | nd | 7440 | 4570 | 1230 |
| 14 | < | 8310 | nd | 2990 | 4880 |
| 15 | < | 10820 | nd | 4160 | 810 |
| 16 | < | 8710 | nd | 5690 | 5600 |
| 17 | < | 7020 | 4480 | 5340 | 670 |
| 18 | < | 8370 | 6250 | 4780 | 370 |
| 19 | < | 8520 | 1600 | 310 | 2330 |

Tb patients, 1-4 month after diagnosis

| | | | | | |
|---|---|---|---|---|---|
| 20 | < | nd | 10670 | 12680 | 9670 |
| 21 | < | nd | 3010 | 1420 | 350 |
| 22 | < | nd | 8450 | 7850 | 1950 |
| 23 | < | 10060 | nd | 3730 | 350 |
| 24 | < | 10830 | nd | 6180 | 320 |
| 25 | < | 9000 | nd | 3200 | 4960 |
| 26 | < | 10740 | nd | 7650 | 1170 |
| 27 | < | 7550 | 6430 | 6220 | 3390 |
| 28 | < | 8090 | 5790 | 4850 | 2095 |
| 29 | < | 7790 | 4800 | 4260 | 1210 |

### Example 3

Species Distribution of rd1-orf5

Presence of rd1-orf5 in Different Mycobacterial Species

In order to determine the distribution of the rd1-ORF5 gene in species belonging to the M. tuberculosis-complex and in other mycobacteria PCR and/or Southern blotting was used. The bacterial strains used are listed in TABLE 5. Genomic DNA was prepared from mycobacterial cells as described previously (Andersen et al. 1992).

TABLE 5
Mycobacterial strains used in this Example.

| | Species and strain(s) | | Source |
|---|---|---|---|
| 1. | M. tuberculosis | H37Rv | ATCC^{a} |
| | | (ATCC 27294) | |
| 2. | | H37Ra | ATCC |
| | | (ATCC 25177) | |
| 3. | | Erdman | Obtained from A. Lazlo, |
| | | | Ottawa, Canada |
| 4. | M. bovis BCG substrain: | | SSI^{b} |
| | Danish 1331 | | |
| 5. | | Chinese | SSI^{c} |
| 6. | | Canadian | SSI^{c} |
| 7. | | Glaxo | SSI^{c} |
| 8. | | Russia | SSI^{c} |
| 9. | | Pasteur | SSI^{c} |
| 10. | | Japan | WHO^{e} |
| 11. | M. bovis MNC 27 | | SSI^{c} |
| 12. | M. africanum | | Isolated from a Danish |
| | | | patient |
| 13. | M. leprae (armadillo- | | Obtained from J. M. |
| | derived) | | Colston, London, UK |
| 14. | M. avium (ATCC 15769) | | ATCC |
| 15. | M. kansasii (ATCC | | ATCC |
| | 12478) | | |
| 16. | M. marinum (ATCC 927) | | ATCC |
| 17. | M. scrofulaceum (ATCC | | ATCC |
| | 19275) | | |
| 18. | M. intracellulare (ATCC | | ATCC |
| | 15985) | | |
| 19. | M. fortuitum (ATCC | | ATCC |
| | 6841) | | |
| 20. | M. xenopi | | Isolated from a Danish |
| | | | patient |
| 21. | M. flavescens | | Isolated from a Danish |
| | | | patient |
| 22. | M. szulgai | | Isolated from a Danish |
| | | | patient |
| 23. | M. terrae | | SSI^{c} |
| 24. | E. coli | | SSI^{d} |
| 25. | S. aureus | | SSI^{d} |

^{a}American Type Culture Collection, USA.
^{b}Statens Serum Institut, Copenhagen, Denmark.
^{c}Our collection Department of Mycobacteriology, Statens Serum Institut, Copenhagen, Denmark.
^{d}Department of Clinical Microbiology, Statens Serum Institut, Denmark.
^{e}WHO International Laboratory for Biological Standards, Statens Serum Institut, Copenhagen, Denmark.

The Southern blotting was carried out as described previously (Oettinger and Andersen, 1994) with the following modifications: 2 jig of genomic DNA was digested with PvuII, electrophoresed in an 0.8% agarose gel, and transferred onto a nylon membrane (Hybond N-plus; Amersham International plc, Little Chalfont, United Kingdom) with a vacuum transfer device (Milliblot, TM-v; Millipore Corp., Bedford, Mass.). The rd1-ORF5 gene fragments were amplified by PCR from the pTO88 by using the primers shown in TABLE 1 (in Example 1). The probes were labelled non-radioactively with an enhanced chemiluminescence kit (ECL; Amersham International plc, Little Chalfont, United Kingdom). Hybridization and detection was performed according to the instructions provided by the manufacturer. The results are summarized in TABLE 6.

TABLE 6
Interspecies analysis of the rd1-ORF5 gene by Southern blotting.

| Species and strain | | rd1-ORF5 |
|---|---|---|
| 1. | M. tub. H37Rv | + |
| 2. | M. bovis | + |
| 3. | M. bovis BCG Danish 1331 | − |
| 4. | M. bovis BCG Japan | − |
| 5. | M. avium | − |
| 6. | M. kansasii | − |
| 7. | M. marinum | − |
| 8. | M. scrofulaceum | − |
| 9. | M. intracellulare | − |
| 10. | M. fortuitum | − |
| 11. | M. xenopi | − |
| 12. | M. szulgai | − |

+, positive reaction;
−, no reaction,
N.D. not determined.

Positive results for rd1-ORF5 were only obtained when using genomic DNA from M. tuberculosis and M. bovis, and not from M. bovis BCG or other mycobacteria analyzed.

### Example 4

Identification of the Immunogenic Portions of RD1-ORF5

Peptide synthesis: The immunological evaluation of recombinant RD1-ORF5 was described in example 2. Thirty-five overlapping peptides covering the complete amino acid sequence of RD1-ORF5 were purchased from Mimotopes Pty Ltd. The peptides were synthesized by Fmoc solid phase strategy. No purification steps were performed. Lyophilised peptides were stored dry until reconstitution in PBS.

| | |
|---|---|
| RD1-ORF5-p1 | MDYFIRMWNQAALAMEVY |
| RD1-ORF5-p2 | AALAMEVYQAETAVNTLF |
| RD1-ORF5-p3 | ETAVNTLFEKLEPMASIL |
| RD1-ORF5-p4 | LEPMASTLDPGASQSTTN |
| RD1-ORF5-p5 | GASQSTTNPIFGMPSPGS |
| RD1-ORF5-p6 | FGMPSPGSSTPVGQLPPA |
| RD1-ORF5-p7 | PVGQLPPAATQTLGQLGE |
| RD1-ORF5-p8 | QTLGQLGEMSGPMQQLTQ |
| RD1-ORF5-p9 | GPMQQLTQPLQQVTSLFS |
| RD1-ORF5-p10 | QQVTSLESQVGGTGGGNP |
| RD1-ORF5-p11 | GGTGGGNPADEEAAQMGL |
| RD1-ORF5-p12 | EEAAQMGLLOTSPLSNHP |
| RD1-ORF5-p13 | TSPLSNHPLAGGSOPSAG |
| RD1-ORF5-p14 | GGSGPSAGAGLLRAESLP |
| RD1-ORF5-p15 | LLRAESLPGAGGSLTRTP |
| RD1-ORF5-p16 | GGSLTRTPLNSQLIEKPV |
| RD1-ORF5-p17 | SQLTEKPVAPSVMPAAAA |
| RD1-ORF5-p18 | SVMPAAAAGSSATGGAAP |
| RD1-ORF5-p19 | ATGGAAPVGAGAMGQGAQ |
| RD1-ORF5-p20 | AMGQGAQSGGSTRPGLVA |
| RD1-ORF5-p21 | TRPGLVAPAPLAQEREED |
| RD1-ORF5-p22 | AQEREEDDEDDWDEEDDW |
| RD1-ORF5-p23 | MLWHAMPPELNTARLMAG |
| RD1-ORF5-p24 | ARLMAGAGPAPMLAAAAG |
| RD1-ORF5-p25 | PMLAAAAGWQTLSAALDA |
| RD1-ORF5-p26 | TLSAALDAQAVELTARLN |
| RD1-ORF5-p27 | VELTARLNSLGEAWTGGG |
| RD1-ORF5-p28 | GEAWTOGGSDKALAAATP |
| RD1-ORF5-p29 | KALAAATPMVVWLQTAST |
| RD1-ORF5-p30 | VWLQTASTQAKTRAMQAT |
| RD1-ORF5-p31 | KTPMQATAQAAAYTQMAM |
| RD1-ORF5-p32 | AAYTQAMATTPSLPEIAA |
| RD1-ORF5-p36 | TPSLPEIAANHTTQAVLT |
| RD1-ORF5-p33 | LPETAANHITQAVLTATN |
| RD1-ORF5-p34 | VLTATNEEGINTIPIALT |
| RD1-ORF5-p35 | NTIPIALTEMDYEIRMWN |

### Example 5

Interferon-γ Release from PBMC Isolated from Human TB Patients and PPD Positive Healthy Donors

Human donors: PBMC were obtained from healthy donors with a positive in vitro response to purified protein derivative (PPD) or from TB patients with microscopy or culture proven infection.

Lymphocyte preparations and cell culture: PBMC were freshly isolated by gradient centrifugation of heparinized blood on Lymphoprep (Nycomed, Oslo, Norway) and stored in liquid nitrogen until use. The cells were resuspended in complete RPMI 1640 medium (Gibco BRL, Life Technologies) supplemented with 1% penicillin/streptomycin (Gibco BRL, Life Technologies), 1% non-essential-amino acids (FLOW, ICN Biomedicals, Calif., USA), and 10% heat-inactivated normal human AB serum (NHS). The viability and number of the cells were determined by Nigrosin staining. Cell cultures were established with 1.25×10⁵ PBMCs in 100 μl in microtitre plates (Nunc, Roskilde, Denmark) and stimulated with 5 μg/ml PPD and with synthetic peptides at concentrations of 1, 2.5 and 10 ug/ml. No antigen (No ag) was used as a negative control, and phytohaemagglutinin (PHA) was used as a positive control. Supernatants for the analysis of secreted cytokines were harvested after 5 days of culture, pooled, and stored at −80° C. until use.

Cytokine analysis: Interferon-γ (IFN-γ) was detected with a standard sandwich ELISA technique using a commercially available pair of monoclonal antibodies (Endogen, Mass., US) and used according to the manufacturer's instructions. Recombinant IFN-γ (Endogen, Mass., US) was used as a standard. All data are means of duplicate wells and the variation between the wells did not exceed 10% of the mean.

The peptides of RD1-ORF5 were tested in PBMC from 3 PPD positive healthy donors and from one person who has been treated for TB previously (T2). The results of IFN-γ stimulation shown in Table 7 revealed a number of immunogenic peptides on RD1-ORF5 which stimulated IFN-γ production to >300 μg/ml in at least one donors. As is expected, due to the genetic heterogenity of the human population, the recognition patterns from the two positive donors are different.

TABLE 7
Stimulation of IFN-γ release (pg/ml) in PBMC by peptides derived
from RD1-ORF5. Responses to PPD are shown for comparison.

| | KTB8 | KTB3 | B23 | T2 |
|---|---|---|---|---|
| NoAg | 17 | 0 | 8 | 0 |
| PPD | >2362 | >1943 | >1976 | >2090 |
| RD1-ORF5-p1 | 2 | 10 | 980 | 0 |
| RD1-ORF5-p2 | 122 | 6 | 980 | 13 |
| RD1-ORF5-p3 | 9 | 0 | 747 | 432 |
| RD1-ORF5-p4 | 0 | 0 | 1062 | 541 |
| RD1-ORF5-p5 | 0 | 89 | 16 | 5 |
| RD1-ORF5-p6 | 31 | 5 | 150 | 0 |
| RD1-ORF5-p7 | 0 | 144 | 249 | 0 |
| RD1-ORF5-p8 | 3 | 50 | 22 | 0 |
| RD1-ORF5-p9 | 0 | 0 | 1186 | 245 |
| RD1-ORF5-p10 | 0 | 0 | 213 | 249 |
| RD1-ORF5-p11 | 0 | 29 | 1102 | 465 |
| RD1-ORF5-p12 | 0 | 0 | 838 | 714 |
| RD1-ORF5-p13 | 0 | 0 | 363 | 2 |
| RD1-ORF5-p14 | 2 | 0 | 178 | 10 |
| RD1-ORF5-p15 | 3 | 0 | 5 | 10 |
| RD1-ORF5-p16 | 1 | 3 | 232 | 3 |
| RD1-ORF5-p17 | 1 | 0 | 1498 | 669 |
| RD1-ORF5-p18 | 7 | 0 | 1569 | 968 |
| RD1-ORF5-p19 | 2 | 7 | 37 | 4 |
| RD1-ORF5-p20 | 0 | 0 | 1643 | 1326 |
| RD1-ORF5-p21 | 0 | 0 | 0 | 37 |
| RD1-ORF5-p22 | 4 | 0 | 1114 | 580 |
| RD1-ORF5-p23 | 108 | 4 | 466 | 76 |
| RD1-ORF5-p24 | 1 | 0 | 1186 | 10 |
| RD1-ORF5-p25 | 0 | 51 | 846 | 18 |
| RD1-ORF5-p26 | 0 | 0 | 187 | 4 |
| RD1-ORF5-p27 | 0 | 0 | 406 | 3 |
| RD1-ORF5-p28 | 0 | 4 | 474 | 3 |
| RD1-ORF5-p29 | 0 | 3 | 125 | 5 |
| RD1-ORF5-p30 | 0 | 0 | 52 | 17 |
| RD1-ORF5-p31 | 0 | 0 | 1071 | 0 |
| RD1-ORF5-p32 | 0 | 0 | 258 | 0 |
| RD1-ORF5-p33 | 58 | 0 | 208 | 0 |
| RD1-ORF5-p34 | 17 | 209 | >2274 | 739 |
| RD1-ORF5-p35 | 0 | 106 | >2067 | 45 |

### Example 6

Cloning of the Genes Encoding Low Mass Proteins from the ESAT-6 Family

The genes encoding Rv2653c or Rv2654c were cloned into the expression vector pMCT3 (identical to pMCT6, Harboe et al, 1998, except that it only contains six N-terminal histidine residues), by PCR amplification with gene specific primers, for recombinant expression in E. coli of the proteins.

For cloning of the proteins, the following gene specific primers were used:

| | |
|---|---|
| Rv2653c: | |
| PA2653c: | 5′-CTGAGATCTTTGACCCACAAGCGCACTAAA (Bg/II). |
| PB2653c: | 5′-CTCCCATGGTCACTGTTTOGOTGTCGGGTTC (NcoI). |
| Rv2654c: | |
| PA2654c: | 5′-CTGAGATCTATGAGCGGCCACGCGTTGGCT (Bg/II). |
| PB2654c: | 5′-CTCCCATGGTCACGGCGGATCACCCCGGTC (NcoI). |

The primers listed above create the restriction sites indicated after each sequence. The restriction sites are used for the cloning in pMCT3. Where an alternative start codon to ATG is used in the original sequence the primers introduce an ATG codon instead. PCR reactions contained 10 ng of M. tuberculosis chromosomal DNA in 1×PCR buffer+Mg (Boehringer Manheim) with 400 μM dNTP mix (Boehringer Mannheim), 0.4 pM of each primer and 1.5 unit Tag DNA polymerase (Boehringer Mannheim) in 50 μl reaction volume. Reactions were initially heated to 94° C. for 5 min., run for 30 cycles of the program; 92° C. for 1 min., 52° C. for 1 min. and 72° C. for 2min. and terminating with 72° C. for 7 min., using PTC-200 thermal cycler (M J Research, Inc.). The PCR products were cloned into the pRC2.1 cloning vector and transformed into One Shot™ E. coil cells (Invitrogen, Leek, The Netherlands) as described by the manufacturer. Plasmid DNA was digested with the appropriate restriction enzymes (see primer sequence) and cloned into pMCT3 and transformed into E. coli XL-1 Blue cells. The correct insert was always confirmed by sequencing. Sequencing of DNA was performed at Statens Serum Institut using the cycle sequencing system in combination with an automated gel reader (model 373A; Applied Biosystems).

Expression and Purification of Recombinant Rv2653c and Rv2654c.

Expression and metal affinity purification of recombinant protein was undertaken essentially as described by the manufacturers. LB-media containing 100 μg/ml ampicillin and 12.5 μg/ml tetracyclin, was inoculated with overnight culture of XL1-Blue cells harbouring recombinant pMCT3 plasmid. The culture was shaken at 37° C. until it reached a density of OD₆₀₀=0.5. IPTG was hereafter added to a final concentration of 1 mM and the culture was further incubated 2-16 hours. Cells were harvested, resuspended in 1×sonication buffer+8 M urea and sonicated 5×30 sec. with 30 sec. pausing between the pulses. After centrifugation, the lysate was applied to a column containing 10 ml Talon resin (Clontech, Palo Alto, USA). The column was washed and eluted as described by the manufacturers.

Fractions containing recombinant protein were pooled and to gain homogenous protein preparations the pooled fractions were subjected to either the multielution technique (Andersen and Heron, 1993) or anion exchange on a Hitrap column (Pharmacia, Uppsala, Sweden).

TABLE 8
List of nucleotide sequences with their name,
Open Reading Frame (ORF) and SEQ ID NOs

| Protein | ORF: | SEQ ID NO: |
|---|---|---|
| Rv2653c | 324 | 1 |
| Rv2654c | 246 | 3 |

TABLE 9
List of proteins with their name, molecular mass (measured
in Daltons), their Isolectric point and their SEQ ID NO's.

| | | Molecular | Isolectric | SEQ |
|---|---|---|---|---|
| Protein | Size (aa) | Mass (Da) | Point | ID NO: |
| Rv2653c | 107 | 12359.82 | 8.20 | 2 |
| Rv2654c | 81 | 7697.71 | 5.04 | 4 |

### Example 7

Interferon-γ Induction of T Cell Lines

The purified recombinant proteins were screened for the ability to induce a T cell response measured as IFN-γ release. The screening involved testing of the IFN-γ induction of T cell lines generated from PPD positive donors and/or a measurement of the response in PBMC preparations obtained from TB patients, PPD positive as well as negative healthy donors.

Human donors: PBMC were obtained from healthy donors with a positive in vitro response to PPD.

T cell line preparation: T cell lines were prepared by culturing 1-5×10⁶ freshly isolated PBMC with viable M. tuberculosis for 1½ hour at a ratio of 5 bacteria per cell in a total volume of 1 ml. After washing, the cells were cultured in RPMI 1640 medium (Gibco, Grand Island, N.Y) supplemented with HEPES, and 10% heat-inactivated NHS. After 7 days in culture at 37° C. and 5% CO₂, T cells were supplemented with 30-50 U/well of r-IL-2 (recombinant interleukin-2) (Boehringer Mannheim) for approximately 7 days. Finally, the T cell lines were tested for reactivity against the recombinant antigen by stimulating 1-5×10⁵ cells/ml with 5 μg/ml of PPD and recombinant Rv2653c in the presence of 5×10⁵ autologous antigen-presenting cells/ml. No antigen (No ag) and PHA were used as negative and positive controls, respectively. The supernatants were harvested after 4 days of culture and stored at −20° C. until the presence of IFN-γ was analysed. Responses obtained with different T cell lines are shown in Table 10, where donor 1 and 2 are based on T cell lines driven by viable M. tuberculosis.

TABLE 10
Stimulation of T cell lines with recombinant antigen. Responses to PHA
and PPD are shown for comparison. Results are presented as pg IFN-γ/ml.

| | | PHA | PPD | Rv2653c |
|---|---|---|---|---|
| Donor | No ag | (1 μg/ml) | (5 μg/ml) | (5 μg/ml, 1 μg/ml) |
| 1 | 350 | 3940 | 3690 | 1283, 853 |
| 2 | 325 | 3845 | 1824 | 673, 270 |

The results shown in Table 10 indicate that Rv2653c antigen can induce IFN-γ production in T-cell lines generated from PPD positive individuals.

### Example 8

Interferon-γ Induction in Human TB Patients and BCG Vaccinated

Human donors: PBMC were obtained from healthy BCG vaccinated donors with no known exposure to M. tuberculosis and from patients with culture or microscopy proven infection with TB. Blood samples were drawn from the TB patients 0-6 months after diagnosis.

Lymphocyte preparations and cell culture: PBMC were freshly isolated by gradient centrifugation of heparinized blood on Lymphoprep (Nycomed, Oslo, Norway) and stored in liquid nitrogen until use. The cells were resuspended in complete RPMI 1640 medium (Gibco, Grand Island, N.Y.) supplemented with 1% penicillin/streptomycin (Gibo BRL, Life Technologies), 1% non-essentiel-amino acids (FLOW, ICN Biomedicals, Calif., USA), and 10% normal human ABO serum (NHS) from the local blood bank. The number and the viability of the cells were determined by Nigrosin staining. Cultures were established with 1.25×10⁵ PBMCs in 50 μl in microtitre plates (Nunc, Roskilde, Denmark) and stimulated with ST-CF, PDD and Rv2653c. No antigen (No ag) and phytohaemagglutinin (PHA) were used as negative and positive control, respectively. Supernatants for the detection of cytokines were harvested after 5 days of culture, pooled, and stored at −80° C. until used.

Cytokine analysis: lnterferon-γ (IFN-γ) was detected with a standard sandwich ELISA technique using a commercially available pair of monoclonal antibodies (Endogen) and used according to the manufacturer's instruction. Recombinant IFN-γ (Endogen) was used as a standard. All data are means of duplicate wells and the variation between wells did not exceed 10% of the mean. Cytokine levels below 50 pg/mI were considered negative. Responses of 42 individual donors are shown in Table 11.

TABLE 11
Stimulation of PBMCs from 9 healthy PPD and/or ST-CF negative,
13 healthy PPD and/or ST-CF positive donors and 6 Tb patients with
recombinant antigen. ST-CF, PPD and PHA are shown for comparison.
Results are given in pg IFN-γ/ml.
Healthy PPD and/or ST-CF negative donors.

| | | | | STCF | Rv2653c | Rv2653c |
|---|---|---|---|---|---|---|
| Donor | no ag | PHA | PPD | (2.5 μg/ml) | (5 μg/ml) | (2.5 μg/ml) |
| A | 0 | 3354 | 113 | nd. | 0 | 4 |
| B | 0 | 3803 | 563 | nd. | 0 | 50 |
| C | 0 | 3446 | 525 | nd. | 97 | 0 |
| D | 32 | 1919 | nd. | 234 | nd | nd. |
| E | 0 | 2889 | nd. | 178 | nd. | nd. |
| F | 42 | 3998 | nd. | 175 | nd. | nd. |
| G | 44 | 6269 | 190 | 195 (5 μg) | nd. | nd. |
| H | 5 | 2282 | n.d. | 10 (5 μg) | nd. | nd. |
| I | 2 | 10427 | n.d. | 80 (5 μg) | nd. | nd. |

Healthy PPD and/or ST-CF positive donors.

| | | | | | | |
|---|---|---|---|---|---|---|
| A | 31 | 6716 | 2275 | nd. | 1 | 62 |
| B | 43 | 4733 | 6159 | nd. | 179 | 126 |
| C | 7 | 6165 | 5808 | nd. | 110 | 30 |
| D | 63 | 6532 | 6314 | nd. | 2445 | 235 |
| E | 14 | 5614 | 3852 | nd. | 147 | 448 |
| F | 13 | 3493 | 4327 | 3381 | nd. | nd. |
| G | 12 | 8164 | nd. | 738 | nd. | nd. |
| H | 5 | 7378 | 840 | nd. | nd. | nd. |
| I | 0 | 5168 | n.d. | 4241 | nd. | nd. |
| J | 12 | 4873 | nd. | 745 | nd. | nd. |
| K | 1 | 4512 | nd. | 2137 | nd. | nd. |
| L | 75 | 8047 | nd. | 2778 | nd. | nd. |
| M | 52 | 6095 | nd. | 9133 | nd. | nd. |

The results shown in Table 11 regarding the recombinant antigen Rv2653c indicate that this antigen can induce IFN-γ production in PBMCs from healthy PPD and/or ST-CF positive individuals and/or Tb patients.

### Example 9

Identification of the Immunogenic Portions of the Two Molecules Rv2653c and Rv2654c

The two proteins, of which we are here identifying the immunogenic portions, were previously identified as part of the esat-6 gene family (example 6 and WO01/04151).

Synthetic overlapping peptides covering the complete amino acid sequence of the two proteins were purchased from Mimotopes Pty Ltd. The peptides were synthesized by Fmoc solid phase strategy. No purification steps were performed. Lyophilised peptides were stored dry until reconstitution in PBS.

RV2653C peptides

| | |
|---|---|
| Rv2653c-p1: | MTHKRTKRQPAIAAGLNA |
| Rv2653c-p2: | AIAAGLNAPRRNRVGRQH |
| Rv2653c-p3: | RNRVGRQHGWPADVPSAE |
| Rv2653c-p4: | PADVPSAEQRRAQRQRDL |
| Rv2653c-p5: | RAQRQRDLEAIRRAYAEM |
| Rv2653c-p6: | IRRAYAEMVATSHEIDDD |
| Rv2653c-p7: | TSHEIDDDTAELALLSMH |
| Rv2653c-p8: | ELALLSMHLDDEQRRLEA |
| Rv2653c-p9: | DEQRRLEAGMKLGWHPYH |
| Rv2653c-p10: | MKLGWHPYHFPDEPDSKQ |

RV2654C peptides

| | |
|---|---|
| Rv2654c-p1: | MSGHALAARTLLAAADEL |
| Rv2654c-p2: | AADELVGGPPVEASAAAL |
| Rv2654c-p3: | ASAAALAGDAAGAWRTAA |
| Rv2654c-p4: | AWRTAAVELARALVRAVA |
| Rv2654c-p5: | LVRAVAESHGVAAVLFAA |
| Rv2654c-p6: | VLFAATAAAAAVDRGDPP |

### Example 10

Biological Activity of the Synthetic Peptides Covering Rv2653c and Rv2654c

The above listed synthetic peptides, covering the protein sequence of Rv2653c and Rv2654c, were screened as single peptides and pools for their ability to induce a T cell response measured as IFN-γ release. The screening involved testing of the IFN-γ induction in PBMC preparations obtained from TB patients and BCG vaccinated healthy donors.

Human donors: PBMC were obtained from 15 healthy BCG vaccinated donors and 8 TB patients with microscopy or culture proven infection. Blood samples were drawn from TB patients 0-6 months after diagnosis.

Lymphocyte preparations and cell culture: PBMC were freshly isolated by gradient centrifugation of heparinized blood on Lymphoprep (Nycomed, Oslo, Norway) and stored in liquid nitrogen until use. The cells were resuspended in complete RPMI 1640 medium (Gibco BRL, Life Technologies) supplemented with 1% penicillin/streptomycin (Gibco BRL, Life Technologies), 1% non-essentiel-amino acids (FLOW, ICN Biomedicals, Calif., USA), and 10% heat-inactivated normal human AB serum (NHS). The viability and number of the cells were determined by Nigrosin staining. Cell cultures were established with 1.25×10⁵ PBMCs in 100 μl in microtitre plates (Nunc, Roskilde, Denmark) and stimulated with 5 μg/ml PPD and single peptide in concentrations of 1 and 5 μg/ml and/or peptide pools in which the final concentrations of each peptide was 5 or 1 μg/ml (Table 12, 13 and 14).

“No antigen” was included as negative control and phytohaemagglutinin (PHA) was used as positive control. Supernatants for the analysis of secreted cytokines were harvested after 5 days of culture, pooled, and stored at −80° C. until use.

Cytokine Analysis:

As shown in Table 12 and 13 stimulation of PBMC from TB patients with peptide and/or peptide pools of Rv2653c and Rv2654c resulted in a marked release of IFN-γ. As is expected, due to the genetical heterogenity of the human population, some of the peptides/peptide pools are however recognized more frequently and to a higher extent than others.

None of the tested peptide pools resulted in IFN-γ release in BCG vaccinated healthy donors (Table 14) which makes Rv2653c and 2654c ideal candidates for discriminating between TB infected and BCG vaccinated donors.

TABLE 12
Stimulation of PBMCs from 8 TB patients with peptide pools.
Responses to PPD and “no antigen” are shown for comparison.
Results are given as pg IFN-γ/ml. The maximal IFN-γ response
of each peptide pool is given.

| Antigen/donor | Pt1 | Pt2 | Pt3 | Pt4 | Pt5 | Pt6 | Pt7 | Pt8 |
|---|---|---|---|---|---|---|---|---|
| No antigen | 76 | 13 | 42 | 256 | 45 | 19 | 342 | 101 |
| PPD | 3795 | 3366 | 3531 | 3449 | 2303 | 3240 | 1510 | 3919 |
| Rv2653c | 310 | 25 | 577 | 1704 | 97 | 209 | 977 | 68 |
| p1, 2, 3, 6 | | | | | | | | |
| Rv2653c | 153 | 0 | 906 | 1248 | 34 | 102 | 1039 | 95 |
| p7, 8, 9, 10 | | | | | | | | |
| Rv2654c | 81 | 65 | 549 | 2571 | 25 | 72 | 560 | 110 |
| p1, 2, 3 | | | | | | | | |
| Rv2654c | 1219 | 144 | 1585 | 1647 | 426 | 716 | 1413 | 352 |
| p4, 5, 6 | | | | | | | | |

TABLE 13
Stimulation of PBMCs from 2 TB patients with single peptides.
Responses to PPD and “no antigen” are shown for comparison.
Results are given as pg IFN-γ/ml. The maximal
IFN-γ response of each peptide pool is given.

| Antigen/donor | Pt3 | Pt4 |
|---|---|---|
| No antigen | 10 | 8 |
| PPD | 12435 | 16852 |
| ESAT-6 | 34 | 18 |
| Rv2653c p1 | 62 | 164 |
| Rv2653c p2 | 7 | 674 |
| Rv2653c p3 | 38 | 1425 |
| Rv2653c p6 | 53 | 593 |
| Rv2653c p7 | 101 | 1003 |
| Rv2653c p8 | 153 | 1160 |
| Rv2653c p9 | 27 | 261 |
| Rv2653c p10 | 61 | 691 |
| Rv2654c p1 | 64 | 2041 |
| Rv2654c p2 | 136 | 522 |
| Rv2654c p3 | 257 | 1004 |
| Rv2654c p4 | 1135 | 3556 |
| Rv2654c p5 | 80 | 955 |
| Rv2654c p6 | 488 | 1736 |

TABLE 14
Stimulation of PBMCs from 10 BCG vaccinated healthy donors with
peptide pools. Responses to PPD and “no antigen”
are shown for comparison.
Results are given as pg IFN-γ/ml. The maximal IFN-γ response
of each peptide pool is given.

| Antigen/donor | BCG1 | BCG2 | BCG3 | BCG4 | BCG5 | BCG6 | BCG7 | BCG8 | BCG9 | BCG10 |
|---|---|---|---|---|---|---|---|---|---|---|
| No antigen | 0 | 26 | 16 | 0 | 5 | 0 | 0 | 10 | 1 | 0 |
| PPD | 14706 | 4103 | 6539 | 8289 | 2516 | 818 | 5041 | 5315 | 859 | 12322 |
| ESAT-6 | 0 | 6 | 36 | 3 | 6 | 0 | 8 | 31 | 0 | 11 |
| Rv2653c | 160 | 1 | 236 | 10 | 8 | 17 | 15 | 38 | 4 | 23 |
| p1, 2, 3, 6 | | | | | | | | | | |
| Rv2653c | 0 | 9 | 8 | 18 | 0 | 0 | 2 | 0 | 5 | 0 |
| p7, 8, 9, 10 | | | | | | | | | | |
| Rv2654c | 0 | 4 | 11 | 0 | 0 | 4 | 0 | 0 | 3 | 0 |
| p1, 2, 3 | | | | | | | | | | |
| Rv2654c | 25 | 0 | 8 | 0 | 1 | 0 | 0 | 0 | 530 | 0 |
| p4, 5 | | | | | | | | | | |

### Example 11

Cloning and Expression of Rv2653c and Rv2654c in E. coli

The coding regions Rv2653c and Rv2654c was amplified by PCR using the following sets of primers:

| | |
|---|---|
| Rv2653-F: | GGGGACAAGTTTGTACAAAAAACCAGGCTTA TTG ACC CAC AAG CCC ACT AA |
| Rv2653-R: | GGGGACCACTTTGTACAAGAAAGCTGGGTCCTA CTG TTT GCT GTC GGG TTC GT |
| Rv2654-F: | GGGGACAAGTTTGTACAAAAAAGCAGGCTTA AGC CGC CAC GCG TTG GC |
| Rv2654-R: | GGGGACCACTTTGTACAAGAAAGCTGCGTCCTA CGG CGG ATC ACC CCC GT |

PCR reactions were carried out using Platinum Tag DNA Polymerase (GIBCO BRL) in a 50 μl reaction volume containing 60 mM Tris-SO₄ (pH 8.9), 18 mM Ammonium Sulfate, 0.2 mM of each of the four nucleotides, 0.2 μM of each primer and 10 ng of M. tuberculosis H37Rv chromosomal DNA. The reaction mixtures were initially heated to 95° C. for 5 min., followed by 35 cycles of: 95° C. for 45 sec, 60° C. for 45 sec and 72° C. for 2 min. The amplification products were precipitated by PEG/MgCl₂, and dissolved in 50 μL TE buffer.

DNA fragments were cloned and expressed in Gateway Cloning system (Life Technology). First, to create Entry Clones, 5 μL of DNA fragment was mixed with 1 μL of DONR201, 2 μL of BP CLONASE enzyme mix and 2 μL of BP reaction buffer. The recombination reactions were carried out at 25° C. for 60 min. After Proteinase K treatment at 37° C. for 10 min., 5 μL of each sample was used to transform E. coli DH5α competent cells. Transformants were selected on LB plates containing 50 μg/mL kanamycin. One bacterial clone from each transformation was grown in 3 mL LB medium containing 50 μg/mL kanamycin and plasmid DNA was isolated (Qiagen).

Second, to create expression clones, 2 μL of each entry clone DNA was mixed with 1 μL of His-tagged expression vector (pDest17), 2 μL LR reaction buffer, 2 μL LR CLONASE enzyme mix and 3 μL TE. After recombination at 25° C. for 60 min. and proteinase K treatment at 37° C. for 10 min., 5 μL of each sample was used to transform E. coli BL21-SI competent cells. Transformants were selected on LBON (LB without NaCl) plates containing 100 μg/mL ampicillin. The resulting E. coli clones express recombinant proteins carrying a 6-histine tag at the N-terminal. All clones were confirmed by DNA sequencing.

To purify recombinant proteins transformed E. coli BL21-SI cells were cultured in 900 mL LBON medium containing 100 μg/mL at 30° C. until OD₆₀₀=0.4-0.6. At this point 100 mL 3 M NaCl was added and 3 hours later bacteria were harvested by centrifugation. Bacteria pellets were resuspended in 20 mL bacterial protein extraction reagent (Pierce) incubated for 10 min. at room temperature and pelleted by centrifugation. Bacteria were lysed and their DNA digested by treating with lysozyme (0.1 mg/mL) and DNase 1 (2.5 μg/mL) at room temperature for 30 min. with gentle agitation. The recombinant proteins form inclusion bodies and were therefore pelleted by centrifugation at 27.000×g for 15 min. Protein pellets were solubilized by adding 20 ml of sonication buffer (8 M urea, 50 mM Na₂HPO₄, 100 mM Tris-HCl, pH 8.0) and sonicate 5×30 sec, with 30 sec pausing between the pulses. After centrifugation at 27.000×g for 15 min., supernatants were applied to 10 mL TALON columns (Clonetech). The columns were each washed with 50 mL sonication buffer. Bound proteins were eluted by lowering pH (8 M urea, 50 mM Na₂HPO₄, 100 mM Tris-HCl, pH 4.5). 5 mL fractions were colleted and analyzed by Coomassie stained SDS-PAGE. Fractions containing recombinant protein were pooled. Further purifications were achieved by anion- or cation-exchange chromatography on Hitrap columns (Pharmacia). Bound proteins were eluted using a NaCl gradient from 0-500 mM in 3 M urea, 10 mM Tris-HCl, pH 8.0. All fractions were colleted and analyzed on SDS-PAGE using Coomassie staining. Fractions containing recombinant protein were pooled. Final protein concentrations were determined by micro BCA (Pierce).

### Example 12

Serological Recognition of Recombinant Rv2653c and Rv2654c

To test the potential of the proteins as serological antigens, sera were collected from 8 TB patients and 4 healthy BCG non-vaccinated controls and were assayed for antibodies recognizing the recombinantly produced proteins in an ELISA assay as follows: Each of the sera were absorbed with Promega E. coli extract (S3761) for 4 hours at room temperature, and the supernatants were collected after centrifugation. 0.5 μg/ml of the proteins in carbonate buffer (pH 9.6) were coated over night at 4° C. to a polystyrene plate (Maxisorp, Nunc). The plates were washed in PBS-0.05% Tween-20 and the sera applied in a dilution of 1:100. After 1 hour of incubation the plates were washed 3 times with PBS-0.05% Tween-20, and 100 ul per well of peroxidase-conjugated Rabbit Anti-Human IgA, IgG, IgM was applied in a dilution of 1:8000 to each well. After 1 hour of incubation the plates were washed 3 times with PBS-0.05% Tween-20. 100 ul of substrate (TMB PLUS, Kem-En-Tec) was added per well, the reaction was stopped after 30 min with 0.2 M Sulphuric acid, and the absorbance was read at 405 nm. The results are shown in table 15.

TABLE 15
Serological recognition of the proteins by TB patients (n = 8) and healthy
controls (n = 4). The percentage of responders as well as the number
of persons responding in each group is indicated. For comparison,
recombinant 38 kDa antigen (r38 kDa, Rv0934) was included in the panel
of recombinant M. tuberculosis proteins investigated. r38 kDa is
considered a promising serological antigen (e.g. Lyashchenko, K.P. et al,
J Immunological Methods 242 (2000) 91-100). The cut-off values for
positive responses are indicated in the table.

| | Percent (n) positive | Percent (n) positive | |
|---|---|---|---|
| Protein | TB patients | healthy controls | Cut off |
| Rv2653c | 100 (8) | 0 (0) | 0.4 |
| Rv2654c | 63 (5) | 0 (0) | 0.3 |
| r38 kDa | 75 (6) | 0 (0) | 0.2 |

As shown in table 15, Rv2653c, Rv2654c and r38kDa are recognized by ≧50% of the TB patients tested. In addition, Rv2653c and Rv2654c were recognized with high OD values (>0.7) by one or more of the TB patients, indicating a particular high amount of specific antibodies to these proteins. None of the proteins are recognized by healthy non-BCG vaccinated controls, which demonstrates the potential of these proteins to differentiate between M. tuberculosis infected individuals and healthy individuals. Rv2653c and Rv2654c are therefore promising serodiagnostic candidates.

References:

Andersen P. et al., 1995, J. Immunol. 154: 3359-72

Andersen P., 1994, Infect. Immun. 62: 2536-44.

Andersen, P. and Heron, 1, 1993, J. Immunol. Methods 161: 29-39.

Andersen, P. et al 1991. Infect. Immun. 59:1905-1910

Andersen, A. B. et al., 1992, Infect. Immun. 60: 2317-2323.

Barkholt, V. and Jensen, A. L., 1989, Anal. Biochem. 177: 318-322.

Borodovsky, M., and J. Mcininch. 1993, Computers Chem. 17:123-133.

Brandt, L., et al. 2000 Infect. Immun. 68:2; 791-795.

Cole, S. T et al 1998 Nature 393: 537-544

Cote-Sierra J, et al 1998, Gene October 9;221 (1):25-34

Danish Patent application PA 1999 01020 (WO 01/23388) “Tuberculosis vaccine and diagnostic based on the Mycobacterium tuberculosis esat-6 gene family”.

Danish Patent application PA 2000 00666 “Nucleic acid fragments and polypeptide fragments derived from M. tuberculosis”

Gosselin et al., (1992) J. Immunol. 149: 3477-3481

Harboe, M. et al., 1996, Infect. Immun. 64: 16-22.

Harboe, M., et al., 1998 Infect. Immun. 66:2; 717-723

Hochstrasser, D. F. et al., 1988, Anal. Biochem. 173: 424-435

Kilgus J et al, J Immunol. 1991 January 1;146(1):307-15

Köhler, G. and Milstein, C., 1975, Nature 256: 495-497.

Li, H. et al., 1993, Infect. Immun. 61: 1730-1734.

Lindblad E. B. et al., 1997, Infect. Immun. 65: 623-629.

Lowry, D. B. et al 1999, Nature 400: 269-71

Luashchenko, K. P., et al 2000. J Immunological Methods 242: 91-100

Lustig et al 1976, Cell Immunol 24(1):164-72

Mahairas, G. G. et al., 1996, J. Bacteriol 178: 1274-1282.

Maniatis T. et al., 1989, “Molecular cloning: a laboratory manual”, 2nd ed., Cold Spring Harbor Laboratory, Cold Spring Harbor, N.Y.

McCafferty et al, Nature, 348:552-554 (1990)

Merrifield, R. B. Fed. Proc. Am. Soc. Ex. Biol. 21: 412,1962 and J. Am. Chem. Soc. 85: 2149, 1963

Mowat et al 1991, Immunology 72(3):317-22

Nagai et al 1991, Infect. Immun 59:1; 372-382

Oettinger, T. and Andersen, A. B., 1994, Infect. Immun. 62: 2058-2064.

Ohara, N. et al., 1995, Scand. J. immunol. 41: 233-442.

Olsen A. W et al, Eur J Immunol. 2000 June; 30(6):1724-32

Pal P. G. and Horwitz M. A., 1992, Infect. Immun. 60: 4781-92.

Patent application U.S. Ser. No. 09/0505,739 “Nucleic acid fragments and polypeptide fragments derived from M. tuberculosis”

Pearson, W. R. and Lipman D. J., 1988. Proc. Natl. Acad. Sci. USA 85: 2444-2448.

Ploug, M. et al., 1989, Anal. Biochem. 181: 33-39.

Pollock. J., et al, 2000. The Veterinary record, 146:659-665

Porath, J. et al., 1985, FEBS Left. 185: 306-310.

Ravn, P. et al 1999. J. Infect. Dis. 179:637-645

Roberts, A. D. et al., 1995, Immunol. 85: 502-508.

Rolph, M. S, and I. A. Ramshaw. 1997. Curr.Opin.Immunol.9:517-24

Rosenkrands, I., et al 1998, Infect. Immun 66:6; 2728-2735

Sambrook et al, Molecular Cloning; A laboratory manual, Cold Spring Harbor Laboratories, NY, 1989

Sinigaglia F et al. Nature 1988 December 22-29;336(6201):778-80

Skjøt, R. L. V. et al 2000, Infect. Immun 68:1; 214-220

Stryhn, A. et al 1996 Eur. J. Immunol. 26:1911-1918

Sørensen, A. L. et al., 1995, Infect. Immun. 63: 1710-1717.

Theisen, M. et al., 1995, Clinical and Diagnostic Laboratory Immunology, 2: 30-34.

Thompson J., et al Nucleic Acids Res 1994 22:4673-4680

Ulmer J. B et al 1993, Curr. Opin. Invest. Drugs 2(9): 983-989

Valdés-Stauber, N. and Scherer, S., 1994, Appl. Environ. Microbiol. 60: 3809-3814.

Valdés-Stauber, N. and Scherer, S., 1996, Appl. Environ. Microbiol. 62: 1283-1286.

van Dyke M. W. et al., 1992. Gene pp. 99-104.

von Heijne, G., 1984, J. Mol. Biol. 173: 243-251.

Williams, N., 1996, Science 272: 27.

Young, R. A. et al., 1985, Proc. Natl. Acad. Sci. USA 82: 2583-2587.

1. A substantially pure polypeptide, which comprises at least one amino acid sequence selected from the group consisting of:
(a) an amino acid sequence selected from Rv2653c, Rv2654c or RD1-ORF5;
(b) an immunogenic portion of any one of the sequences in (a); and
(c) an amino acid sequence analogue having at least 70% sequence identity to any one of the sequences in (a) or (b) and at the same time being immunogenic.

2. A substantially pure polypeptide according to claim 1, wherein the amino acid sequence analogue has at least 80% sequence identity to any of the sequences in (a) or (b).

3. A fusion polypeptide, which comprises at least one amino acid sequence according to claim 1 and at least one fusion partner.

4. A fusion polypeptide according to claim 3, wherein the fusion partner comprises a digit polypeptide fragment selected from the group consisting of:
(a) a polypeptide fragment derived from a virulent mycobacterium;
(b) a polypeptide according to claim 1; and
(c) at least one immunogenic portion of any of such polypeptides in (a) or (b).

5. A polypeptide, which comprises at least one amino acid sequence according to claim 1 which is lipidated so as to allow a self-adjuvating effect of the polypeptide.

6. An immunogenic composition comprising at least one polypeptide according to claim 1.

7. An immunogenic composition according to claim 6, which is in the form of a vaccine.

8. An immunogenic composition according to claim 6, which is in the form of a skin test reagent.

9. A nucleic acid fragment in isolated form which
(a) comprises at least one nucleic acid sequence which encodes a polypeptide as defined in claim 1, or comprises a nucleic acid sequence complementary thereto; and/or
(b) has a length of at least 10 nucleotides and hybridizes under stringent hybridization conditions with a nucleotide sequence selected from Rv2653c, Rv2654c or RD1-ORF5, or a nucleotide sequence complementary to any one of these sequences; or with a nucleotide sequence selected from a sequence in (a).

10. A nucleic acid fragment according to claim 9, which is a DNA fragment.

11. A replicable expression vector, which comprises at least one nucleic acid fragment according to claim 9.

12. A transformed cell harbouring at least one vector according to claim 11.

13. A method for producing a polypeptide according to claim 1, comprising:
(a) inserting a nucleic acid fragment according to claim 12 into a vector which is able to replicate in a host cell, introducing the resulting recombinant vector into the host cell, culturing the host cell in a culture medium under conditions sufficient to effect expression of the polypeptide, and recovering the polypeptide from the host cell or culture medium;
(b) isolating the polypeptide from a whole mycobacterium from culture filtrate or from lysates or fractions thereof; or
(c) synthesizing the polypeptide.

14. A method of diagnosing tuberculosis caused by virulent mycobacteria in an animal, including a human being, comprising intradermally injecting, in the animal, at least one polypeptide according to claim 1 or an immunogenic composition according to claim 6, a positive skin response at the location of injection being indicative of the animal having tuberculosis, and a negative skin response at the location of injection being indicative of the animal not having tuberculosis.

15. A method for immunising an animal, including a human being, against tuberculosis caused by virulent mycobacteria comprising administering to the animal at least one polypeptide according to claim 1 or an immunogenic composition according to claim 6.

16. A monoclonal or polyclonal antibody, which is specifically reacting with a polypeptide according to claim 1 in an immuno assay, or a specific binding fragment of said antibody.

17. A pharmaceutical composition which comprises an immunologically responsive amount of at least one member selected from the group consisting of:
(a) a polypeptide selected from Rv2653c, Rv2654c or RD1-ORF5, or an immunogenic portion thereof;
(b) an amino acid sequence which has a sequence identity of at least 70% to any one of said polypeptides in (a) and is immunogenic;
(c) a fusion polypeptide comprising at least one polypeptide or amino acid sequence according to (a) or (b) and at least one fusion partner;
(d) a nucleic acid sequence which encodes a polypeptide or amino acid sequence according to (a), (b) or (c);
(e) a nucleic acid sequence which is complementary to a sequence according to (d);
(f) a nucleic acid sequence which has a length of at least 10 nucleotides and which hybridizes under stringent conditions with a nucleic acid sequence according to (d) or (e); and
(g) a non-pathogenic micro-organism which has incorporated therein a nucleic acid sequence according to (d), (e) or (f) in a manner to permit expression of a polypeptide encoded thereby.
